(19) United States
(12) Patent Application Publication
(10) Pub. No.: US 2014/0328840 A1
(43) Pub. Date: Nov. 6, 2014

(54) MARKER AND TARGET FOR RESPONSIVENESS AND RESISTANCE TO CANCER AGENTS

(71) Applicants: The Provost, Fellows, Foundation Scholars, and the Other Members of the Board, of the College of the, (unknown); Holy and Undivided Trinity of Queen Elezabeth, Near Dublin, Dublin (IE)

(72) Inventors: Lorraine O'Driscoll, Dublin (IE); Sweta Rani, Dublin (IE)

(73) Assignee: The Provost, Fellows, Foundation Scholars, and the Other Members of the Board, of the College of the, Dublin (IE)

(21) Appl. No.: 14/352,260

(22) PCT Filed: Oct. 22, 2012
(86) PCT No.: PCT/EP2012/070901

§ 371 Date: Apr. 16, 2014

(30) Foreign Application Priority Data
Oct. 24, 2011 (EP) 1118205.2
Nov. 2, 2011 (EP) 1118899.2

(51) Int. Cl. G01N 33/574; C07K 16/26; A61K 39/395; A61K 45/06; A61K 31/713; C12N 15/113; C12Q 1/68

(52) U.S. Cl. CPC G01N 33/57488; C12N 15/1136; C07K 16/26; C12Q 1/6886; G01N 33/57415; A61K 45/06; A61K 31/713; A61K 39/3955
USPC 424/133.1; 514/44 A; 424/139.1; 435/6.11; 435/7.92

(57) ABSTRACT

An assay of identifying the responsiveness of a HER2-positive cancer in a patient to a HER2 targeted drug, the method comprising the step of comparing a level of NeuromedinU in a biological sample obtained from the patient with a reference level of NeuromedinU, wherein detection of a level of NeuromedinU that is increased compared to the reference level of NeuromedinU indicates that the HER2-positive cancer has reduced responsiveness to a HER2 targeted drug.

### FIELD OF THE INVENTION

The invention relates to the novel use of a marker, NeuromedinU (NMU), that has clinical potential as a target and poor-prognostic biomarker for cancer that is directly associated with responsiveness and/or resistance to certain cancer targeting agents.

### BACKGROUND OF INVENTION

Herceptin-2 is currently used to treat Human Epidermal growth factor Receptor 2 (HER2)-positive cancers. HER2 is also known as ErbB-2. This family of growth factor receptors also includes 3 other members, HER1 (EGFR); HER3 and HER4. HER2 is over-expressed in approximately 25% of breast cancers and is associated with higher aggressiveness and poor prognosis. This over-expression, however, is not limited to breast cancer and has been identified in a variety of cancer types including cancers of the bladder, pancreas, non-small cell lung cancer (NSCLC), ovary, colon, kidney, head & neck, stomach, prostate, gliomas, and biologically aggressive forms of uterine cancer, such as uterine serous endometrial carcinoma. Abnormal levels of other HER family members, especially EGFR, are also associated with cancerous states.

Trastuzumab (Herceptin®; Genentech/Roche; targeting HER2) and more recently Lapatinib (Tykerb®; GSK; targeting both HER2+EGFR) have improved prognosis for patients with HER2-positive breast cancer. More recent drugs developed against HER2 include Neratinib (Phase III trials; Pfizer/Puma Biotechnology; an irreversible pan-ErbB receptor tyrosine kinase inhibitor; targeting both HER2+EGFR) and Afatinib (Tovok®; Boehringer Ingelheim; targeting both HER2+EGFR).

Unfortunately, not all HER2-positive patients respond to HER2-targeted or dual HER2/EGFR-targeted agents and others, who initially benefit, relapse due to development of resistance to treatment. Therefore, there is a need to identify biomarkers (ideally minimally-invasive i.e. extracellular also, if possible) for improved patient selection and to develop strategic to improve response and overcome resistance in HER2/EGFR-positive cancers.

Neuromedin U (NmU) has previously been associated with cancer, but an association between NmU and breast cancer or with HER2-overexpression in any cancer type has not previously been established. Specifically, in AML, using K562 cell line expressing a dominant-negative of c-myb proto-oncogene as a model, increased NmU (and NmU-R1) expression was implicated as a growth-promoting autocrine loop. In ovarian cancer, microarray profiling of cell lines derived from ovarian tumour (n=11) and normal (n=2) epithelial cells identified NmU as up-regulated in cancer cells. In pancreatic cancer, immunohistochemistry staining showed significantly higher levels of NmU and NmU-R2 in ducts from pancreatic tumours (n=6) compared to normal pancreas (n=3) or chronic pancreatitis (n=3) ducts. Furthermore, serum NmU levels were significantly lower one week post-major pancreatic resection compared to pre-operative levels. In lung cancer, immunohistochemistry staining of a NSCLC tumour tissue microarray showed NmU expression (n=220 positive; n=106 negative) to be associated (p=0.036) with reduced survival times, and with both increased growth rate and colony formation in lung cancer cell lines. In bladder cancer, NmU over-expression significantly promoted tumour formation of both T24 and T24T cell lines and significantly enhanced T24T lung metastasis. Using RCC10 as cell line model, NmU has recently been found to stimulate migration of renal cancer cell line and to be controlled by von Hippel-Lindau tumour suppressor gene. Conversely, in oesophageal squamous cell carcinoma, reactivation of genes that has been epigenetically silenced in 3 cells lines identified NmU as a potential tumour suppressor gene. In oral cancer, expression profiling of matched tumour and normal specimens (n=5 each), showed NmU as one of 15 mRNAs whose expression levels were down in oral cancer.

It is an object of the present invention to overcome at least one of the above-mentioned problems.

### SUMMARY OF THE INVENTION

Broadly, the invention provides a method of identifying the responsiveness of a HER2-positive cancer in a patient to a HER2 targeted drug, the method comprising the step of comparing a level of NeuromedinU in a biological sample obtained from the patient with a reference level of NeuromedinU, wherein a level of NeuromedinU that is increased compared to the reference level of NeuromedinU indicates that the HER2-positive cancer has reduced responsiveness to a HER2 targeted drug.

The invention also provides a method of identifying the responsiveness of a HER2-positive breast cancer in a patient to a HER2 targeted drug selected from Trastuzumab, Lapatinib, Neratinib, and Afatinib, the method comprising the step of comparing a level of NeuromedinU in a biological sample obtained from the patient with a reference level of NeuromedinU in a HER2-positive cancer that is responsive to the HER2 targeted drug, wherein detection of a level of NeuromedinU that is increased compared to the reference level of NeuromedinU indicates that the HER2-positive cancer has reduced responsiveness to a HER2 targeted drug.

The invention also provides a method of identifying an aggressive HER2-positive cancer in a patient, the method comprising the step of comparing a level of NeuromedinU in a biological sample obtained from the patient with a reference level of NeuromedinU, wherein detection of a level of NeuromedinU that is increased compared to the reference level of NeuromedinU indicates that the HER2-positive cancer is aggressive.

The invention also provides a method of assessing the metastatic potential of a HER2-positive cancer in a patient, the method comprising the step of comparing a level of NeuromedinU in a biological sample obtained from the patient with a reference level of NeuromedinU, wherein detection of a level of NeuromedinU that is increased compared to the reference level of NeuromedinU indicates that the HER2-positive cancer has greater potential for metastasis that a HER2-positive cancer that does not have an increased level of NeuromedinU.

The invention also provides a method of prediction of poor outcome in a patient having a HER2-positive cancer, the method comprising the step of comparing a level of NeuromedinU in a biological sample obtained from the patient with a reference level of NeuromedinU, wherein detection of a level of NeuromedinU that is increased compared to the reference level of NeuromedinU indicates poor outcome for the patient.

The invention also provides a method for the treatment or prevention of a HER2-positive cancer in a patient comprising a step of administering to the patient a therapeutically effective amount of an inhibitor of NeuromedinU. In one embodiment, the patient is identified as having an elevated level of NeuromedinU. Thus, in one embodiment, the method of the invention involves first identifying the NeuromedinU levels of a patient with a HER2-positive cancer, and wherein the patient is identified as having an elevated level of NeuromedinU compared to a reference (i.e. the level of NeuromedinU in a HER2-positive cancer patient that is responsive to HER2 targeting drugs), then treating the patient with a NeuromedinU inhibitor.

The invention also provides a method for the treatment or prevention of a HER2-positive cancer in a patient comprising a step of administering to the patient a therapeutically effective amount of an inhibitor of NeuromedinU and a HER2 targeted drug. In one embodiment, the patient is identified as having an elevated level of NeuromedinU. In another embodiment, the patient is resistant to HER2 targeted drugs. Thus, in one embodiment, the method of the invention involves first identifying the NeuromedinU levels of a patient with a HER2-positive cancer, and wherein the patient is identified as having an elevated level of NeuromedinU compared to a reference (i.e. the level of NeuromedinU in a HER2-positive cancer patient that is responsive to HER2 targeting drugs), then treating the patient with a NeuromedinU inhibitor and a HER2 targeted drug.

The invention also provides a method for increasing the responsiveness of a HER2-positive cancer in a patient comprising a step of administering to the patient a therapeutically effective amount of an inhibitor of NeuromedinU. Typically, the patient is resistant to HER2 targeted drugs.

The invention also provides an inhibitor of NeuromedinU for use as a medicament.

The invention also provides an inhibitor of NeuromedinU for use in a method for the treatment or prevention of a HER2-positive cancer in a patient.

The invention also provides a pharmaceutical composition comprising an inhibitor of NeuromedinU and a pharmaceutically acceptable carrier.

The invention also provides a pharmaceutical composition comprising an inhibitor of NeuromedinU, a HER2 targeted drug, and a pharmaceutically acceptable carrier.

The invention also provides a method for decreasing the responsiveness of a HER2-positive cancer to a HER2 targeted drug in a cell, the method comprising the step of administering to the cell an effective amount of NeuromedinU or a nucleic acid encoding NeuromedinU.

The invention also provides a method for identifying an agent capable of treating or preventing HER2-positive cancer in a patient, the method comprising the steps of providing a cell capable of expressing NeuromedinU, administering a test agent to the cell, and detecting the level of expression of NeuromedinU in the cell compared with an untreated cell, wherein a test agent that causes a reduction in the level of expression of NeuromedinU compared to an untreated cell is an agent capable of treating or preventing HER2-positive cancer.

The invention also provides a method for identifying an agent capable of treating or preventing HER2-positive cancer in a patient, the method comprising the steps of providing a cell capable of expressing NeuromedinU, administering to the cell a HER2 targeting agent, subsequently administering to the cell a test agent, and detecting the level of expression of NeuromedinU in the cell treated with the test agent compared with a cell not treated with the test agent, wherein a test agent that causes a reduction in the level of expression of NeuromedinU compared to an untreated cell is an agent capable of treating or preventing HER2-positive cancer.

The invention also provides a method for identifying an agent capable of increasing the responsiveness of a HER2-positive cancer in a patient to a HER2 targeted drug, the method comprising the steps of providing a cell capable of expressing NeuromedinU, administering a test agent to the cell, and detecting the level of expression of NeuromedinU in the cell compared with an untreated cell, wherein a test agent that causes a reduction in the level of expression of NeuromedinU compared to an untreated cell is an agent capable of increasing the responsiveness of a HER2-positive cancer in a patient to a HER2 targeted drug.

The invention also provides a method for identifying an agent capable of increasing the responsiveness of a HER2-positive cancer in a patient to a HER2 targeted drug, the method comprising the steps of providing a cell capable of expressing NeuromedinU, administering to the cell a HER2 targeting agent, subsequently administering to the cell a test agent, and detecting the level of expression of NeuromedinU in the cell treated with the test agent compared with a cell not treated with the test agent, wherein a test agent that causes a reduction in the level of expression of NeuromedinU compared to an untreated cell is an agent capable increasing the responsiveness of a HER2-positive cancer in a patient to a HER2 targeted drug.

The invention also provides a neuropeptide from the group comprising NeuromedinU and isoforms thereof as a cellular marker for responsiveness and resistance to cancer targeting agents.

The invention also provides a neuropeptide from the group comprising NeuromedinU and isoforms thereof as a target for developing cancer targeting agents to target HER expressing cancers.

The invention also provides a predictive kit and/or assay for determining the responsiveness and/or resistance of tumors to cancer targeting agents comprising a peptide selected from the group comprising NeuromedinU and isoforms thereof.

The invention also provides a cell-based, minimally-invasive blood based method of determining if a patient is responding to and/or resistant to cancer targeting agents comprising taking a sample from a patient and determining the correlation levels of a peptide selected from the group comprising NeuromedinU or isoforms thereof or combinations thereof in the sample, an elevated level thereof, compared to the level in a control sample from an healthy individual, indicating the responsiveness of the cancer targeting agent.

The invention also provides a method as described above wherein the tumour expresses a molecule that is specifically targeted by cancer targeting agents.

In embodiment, the method described above, where any tumor over expresses a group of cell surface protein receptors comprising the human epidermal growth factor receptors (HERs), HER1 or EGFR, HER2, HER3 and HER4.

The invention also provides any cancer agent targeting a tumor expressing one or more of the following factors; HER1 or EGFR, HER2, HER3 and HER4.

In one embodiment, the methods as described above whereby the increased levels of a peptide selected from the group comprising NeuromedinU or isoforms thereof or combinations thereof are directly correlated to poor responsiveness and/or resistance to cancer targeting agents.

In one embodiment, the cancer targeting agents are, but not limited to Trastuzumab (Herceptin®; targeting HER2), Lapatinib (Tykerb®; targeting both HER2+EGFR), Neratinib (targeting both HER2+EGFR), and Afatinib (Tovok®; targeting both HER2+EGFR).

The invention also provides a method of predicting the responsiveness and/or resistance of cancer targeting agents as described above using a neuropeptide from the group comprising NeuromedinU and isoforms thereof.

The invention also provides a method of using NeuromedinU or isoforms thereof and/or combinations thereof as therapeutic targets in the development of alternative or more effective cancer targeting agents. Optionally, the cancer targeting agents as specifically targeting NeuromedinU or isoforms thereof or combinations thereof on tumors expressing one or more of the following factors; HER1 or EGFR, HER2, HER3 and HER4.

In one embodiment, any tumor as described above that over-expresses HER2 and/or EGFR is a cancer type such a breast, bladder, pancreas, NSCLC, ovarian, colon, kidney, head & neck, stomach, prostate, gliomas, biologically aggressive forms of uterine cancer, such as uterine serous endometrial carcinoma.

It is an objection of the present invention to provide a biomarker for the prediction of successful responsiveness of specific chemotherapeutic agents for the treatment of cancer. A further object of the present invention is to meet the increasing demand for improved treatment of cancers and to facilitate personalised medical treatment. A further object is to enable the determination of the success rate of a particular treatment on a patient. Biomarkers could, at least in part, help to overcome the problem of patients receiving certain chemotherapeutic agents from which they derive no benefit, and also meet the increasing demand for improved patient outcomes. A further object of the invention is to provide a target for NeuromedinU signaling as a useful therapeutic strategy for treatment of cancer.

With increasing numbers of personalised drugs entering the market, is has become important to determine the success rate of a particular treatment on a patient.

### BRIEF DESCRIPTION OF DRAWINGS

The invention will be more clearly understood from the following description of an embodiment thereof, given by way of example only, with reference to the accompanying drawings, in which:—

FIG. 1 illustrates significantly higher levels of NmU mRNA detected in culture medium (CM) which reflects the differential levels of expression in the Lapatinib-conditioned compared to Lapatinib-naïve cells, an event initiated shortly after drug exposure. A, Magnitude of fold difference in concentration of Lapatinib that inhibits proliferation of HER2-overexpressing cells that were conditioned with Lapatinib (SKBR3-LR and HCC1954-LR) compared to their aged parent populations (SKBR3-Ag and HCC1954-Ag). B, qPCR analysis of NmU mRNA in CM from (i) SKBR3-LR and (ii) HCC1954-LR compared to CM from their aged-parent cells and corresponding NmU mRNA levels within associated cell populations ((iii) & (iv)). C, qPCR analysis of NmU mRNA following short-term (48 hours) exposure of the parent cell populations SKBR3 and HCC1954 to Lapatinib showing NmU mRNA levels in their respective CM ((i) & (ii) and in the corresponding cells (iii) & (iv)). D, qPCR analysis of NmU mRNA following short-term exposure of the parent cell populations (HCC1954, as example), to (i) Trastuzumab, (iii) Neratinib, and (v) Afatinib showing and NmU mRNA levels in the CM ((i), (iii) & (v)) and in the corresponding cells ((ii), (iv) & (vi)). Data are presented as fold change assigning control an arbitrary 1. All results represent biological repeats n=3±SEM, where *=p<0.05, **=p<0.01, ***=p<0.001.

FIG. 2 illustrates increased NmU mRNA levels are reflected in significantly increased NmU protein expression in cells with acquired-resistance to other HER-targeted agents, Trastuzumab and Neratinib, as well as Lapatinib and also in innately resistant versus sensitive cells. A(i), Higher levels of NmU protein, analysed by ELISA, were also found in (i) Lapatinib-resistant (SKBR3-LR), Trastuzumab-resistant (SKBR3-TR) and Neratinib-resistant (SKBR3-NR) SKBR3 cells compared to the corresponding SKBR3-Ag cells. A(ii), for HCC1954, the same trend was found with Lapatinib-resistant (HCC1954-LR) and Neratinib-resistant (HCC1954-NR) cells compared to their control HCC1954-Ag cells (Trastuzumab-resistant HCC1954 cells are not available to evaluate). Data are presented as NmU protein quantity relative to the amount in the control (100%) population. B, Increased levels of NmU protein were also associated with innate resistance to HER-targeted drugs. Results represent biological repeats n=3±SEM, where *=p<0.05, **=p<0.01, ***=p<0.001.

FIG. 3 illustrates NmU expression is prognostic for poor outcome for breast cancer patients, particularly within the HER2-positive and luminal A molecular subtypes. A, Kaplan-Meier estimates indicate that high levels of NmU are associated with poor prognosis in breast cancer (n=3,489). The association of NmU expression with patients outcome in relation to each breast cancer molecular subtype was subsequently investigated and shown to be significant for B, HER2-positive (n=476) and C, luminal A (n=1,521) tumours (but not C, luminal B (n=676) and D, basal-like (n=454)). E, multivariate analysis reporting on NmU following adjustment for a range of established clinicopathological parameters indicated its independence as a poor prognostic biomarker.

FIG. 4 illustrates NmU over-expression reduces sensitivity to HER-targeted agents, Lapatinib, Trastuzumab, Neratinib and Afatinib. Following NmU cDNA over-expression A, levels of NmU mRNA (qPCR) protein (ELISA) detected in (i) SKBR3 and (ii) HCC1954 cells compared to mock-transfected populations. B, Magnitude of fold difference in concentration of (i) Lapatinib, (ii) Trastuzumab, (iii) Neratinib and (iv) Afatinib that inhibits (by 50%) proliferation of SKBR3-NmU and HCC1954-NmU cells compared to the mock-transfected control cells. Results represent n=3±SEM, where *=p<0.05, **=p<0.01, ***=p<0.001.

FIG. 5 illustrates NmU knock-down partly restores sensitivity to Lapatinib, Trastuzumab, Neratinib and Afatinib in cells with either acquired or innately resistance to HER-targeting. Following transfection with two siRNA targeted to NmU (NmU-1 or NmU-2) or a scrambled sequence (SCR), A(i), qPCR analysis of NmU (adjusted with β-actin) and A(ii) ELISA analysis of NmU protein showed partial knock-down of NmU expression in both the acquired Lapatinib-resistance SKBR3-LR and HCC1954-LR and innately unresponsive T47D and MDA-MB-361 cells. B, NmU knock-down in SKBR3-LR, HCC1954-LR, T47D and MDA-MB-361 cells enhanced the effectiveness of Lapatinib, Trastuzumab, Neratinib and Afatinib at decreasing proliferation compared to that observed in corresponding SCR-transfected cells. Results represent n=3±SEM, where *=p<0.05, **, p<0.01, ***=p<0.001.

FIG. 6 illustrates the mechanism by which NmU affects response to HER-targeted drugs apparently involves changes in expression levels of HER2 target and phosphorylation of EGFR. NmU knockdown with two siRNA (NmU-1 or NmU-2) compared to transfection with a scrambled sequence (SCR) in both SKBR3-LR and HCC1954-LR cells was associated with A, significantly reduced levels of HER2 protein, as shown by (i) ELISA and (ii) immunoblotting. B, Phosphorylation of the remaining HER2 protein seemed to be compromised; but this was not found consistently with two siRNAs. C, Total EGFR (HER1) levels ((i) & (ii)) was not affected in response to NmU knock-down; however, D, phosphorylation (activation) of the EGFR present was significantly reduced. E, NmU knock-down with associated reduced levels of HER2 and p-EGFR was associated with increased levels of phosphorylation Akt. Results represent n=3±SEM, where *=p<0.05, **=p<0.01, ***=p<0.001.

FIG. 7 illustrates exogenous NmU treatment of SKBR3 and HCC1954 parent cells stimulates HER2 and EGFR expression and is associated with low level resistance to HER-targeted drugs. A, NmU-R1 and NmU-R2 receptors were found to be expressed by the parent cells populations using immunoblotting. Treatment with exogenous NmU (24, 48 hours) increased expression of both HER2 and EGFR. B, in SKBR3 cells, increased levels of p-EGFR were also observed. C, Treatment with exogenous NmU (48 hours) conferred a low, but significant level of resistance to Lapatinib (Lap), Trastuzumab (Tra), Neratinib (Ner) and Afatinib (Afa). Results represent n=3±SEM, where *=p<0.05, **=p<0.01, ***=p<0.001.

FIG. 8 illustrates NmU expression is also associated with other phenotypic changes including increased cell motility, invasion and resistance to anoikis. Showing HCC1954 NmU-overexpression and HCC1954-LR NmU-knockdown data as representative results, phenotypic changes, including A, motility, assessed by wound-healing assay (image of cells at range of time points post-wound scratch; below quantitative assessment of same) B, migration, through transwells; C, invasion, through ECM-coated transwells; and D, sensitivity/resistance to anoikis when cultured on p-HEMA to prevent cell attachment were found to be significantly changed as a consequence of NmU manipulation. Results represent n=3±SEM, where *=p<0.05, *=p<0.01, ***=p<0.001.

FIG. 9 illustrates SKBR3 analysis confirmed NmU-overexpression to be associated with stimulating increased cell motility, invasion and anoikis resistance. As shown for HCC1954 (FIG. 51), NmU-overexpression in SKBR3 cells also showed significantly increased A, motility, assessed by wound-healing assay (image of cells at range of time points post-wound scratch; quantitative assessment of same) B, migration, through transwells; C, invasion, through ECM-coated transwells; and D, anoikis resistance. Results represent n=3±SEM, where *=p<0.05, *=p<0.01, ***=p<0.001.

### DETAILED DESCRIPTION OF THE DRAWINGS

### Definitions

In this specification, the term “HER2-positive cancer” should be understood to mean a cancer that overexpresses the HER2 receptor. Examples of HER2-positive cancers are well know by a person skilled in the art, and include breast, NSCLC, pancreas, ovarian, colon, kidney, head and neck, stomach, prostate, gliomas, and biologically aggressive forms of uterine cancer.

In this specification, the term “HER2 targeted drug” should be understood to mean drugs that target the HER2 receptor in mammals, especially humans. Examples of HER2 targeted drugs include Trastuzumab (which is sold by Genentech under the brand name HERCEPTIN®), Lapatinib (which is sold by GSK under the brand name TYKERB®), Neratinib (made by Pfizer under the name HK1-272), and Afatinib (BIBW 2922—Beohringer Ingelheim).

In this specification, the term “NeuromedinU” and “NmU” should be understood to mean a secreted neuropeptide that is synthesised as a 174 amino acid precursor and cleaved to a 25 amino acid biologically-active peptide. The sequence of the active peptide and mRNA encoding the peptide are provided below:

NEUROMEDINU PRECURSOR AA
(SEQ ID NO: 5)
MLRTESCRPRSPAGQVAAASPLLLLLLLLAWCAGACRGAPILPQGLQPEQ
QLQLWNEIDDTCSSFLSIDSQPQASNALEELCFMIMGMLPKPQEQDEKDN
TKRFLFHYSKTQKLGKSNVVSSVVHPLLQLVPHLHERRMKRFRVDEEFQS
PFASQSRGYFLFRPRNGRRSAGFI
NEUROMEDINU ACTIVE PEPTIDE AA
(SEQ ID NO: 1)
FRVDEEFQSPFASQSRGYFLFRPRN
-ref\NM_006681.2\ Homo sapiens NeuromedinU (NmU),
mRNA
(SEQ ID NO: 2)
AGTCCTGTGTCCGGGCCCCGAGGCACAGCCAGGGCACCAGGTGGAGCACC
AGCTACGCGTGGCGCAGCGCAGCGTCCCTAGCACCGAGCCTCCCGCAGCC
GCCGAGATGCTGCGAACAGAGAGCTGCCGCCCCAGGTCGCCCGCCGGACA
GGTGGCCGCGGCGTCCCCGCTCCTGCTGCTGCTGCTGCTGCTCGCCTGGT
GCGCGGGCGCCTGCCGAGGTGCTCCAATATTACCTCAAGGATTACAGCCT
GAACAACAGCTACAGTTGTGGAATGAGATAGATGATACTTGTTCGTCTTT
TCTGTCCATTGATTCTCAGCCTCAGGCATCCAACGCACTGGAGGAGCTTT
GCTTTATGATTATGGGAATGCTACCAAAGCCTCAGGAACAAGATGAAAAA
GATAATACTAAAAGGTTCTTATTTCATTATTCGAAGACACAGAAGTTGGG
CAAGTCAAATGTTGTGTCGTCAGTTGTGCATCCGTTGCTGCAGCTCGTTC
CTCACCTGCATGAGAGAAGAATGAAGAGATTCAGAGTGGACGAAGAATTC
CAAAGTCCCTTTGCAAGTCAAAGTCGAGGATATTTTTTATTCAGGCCACG
GAATGGAAGAAGGTCAGCAGGGTTCATTTAAAATGGATGCCAGCTAATTT
TCCACAGAGCAATGCTATGGAATACAAAATGTACTGACATTTTGTTTTCT
TCTGAAAAAAATCCTTGCTAAATGTACTCTGTTGAAAATCCCTGTGTTGT
CAATGTTCTCAGTTGTAACAATGTTGTAAATGTTCAATTTGTTGAAAATT
AAAAAATCTAAAAATAAA

The methods of the invention employ a step in which NeuromedinU levels in a biological sample from the patient are compared with a reference value/level/abundance. NeuromedinU levels may be determined at a protein or nucleic acid level. For example, the levels of the peptide or precursor protein may be determined using established techniques, or level of the mRNA encoding NeuromedinU may be determined. Methods for determination are described below, and will be well known to those skilled in the art.

In this specification, the term “biological sample” should be understood to mean tumor cells, tumor tissue, conditioned media, blood or blood derivatives (serum, plasma etc), urine, or cerebrospinal fluid.

In this specification, the term “reference level” as applied to NeuromedinU should be understood to mean a level of NeuromedinU detected in a patient identified as having a HER2 positive cancer that is responsive to a HER2 targeted drug, ideally responsive to one or more of Trastuzamab, Lapatinib, Neratinib and Afatinib.

In this specification, the term “reduced responsiveness” should be understood to mean a level of responsiveness that is less than the level of responsiveness in a HER2-positive patient that is responsive to HER2 targeted drugs. The term also may be taken to mean resistance to HER2 targeted drugs in patients with HER2-positive cancers or tumours, either complete resistance or partial resistance. Methods of determining responsiveness or resistance to HER2 targeted drugs or therapies will be known to those skilled in the art and are described below.

In this specification, the term “aggressive” as applied to a HER2-positive cancer is an art-recognised term, and should be understood to mean a HER2-positive cancer that exhibits a degree of movement, invasion, and/or resistance to anoikis, that is comparable to a metastatic HER2-positive cancer cell. The term should be understood to mean a HER2-positive cancer that has elevated potential for metastasis.

In this specification, the term “poor outcome” should be understood to mean that the chances of disease free survival are low.

In this specification, the term “inhibitor of NeuromedinU” should be understood to mean an agent that is capable of decreasing the activity of NeuromedinU in vivo. The activity may be decreased in a number of different ways which will be apparent to a person skilled in the art, including reducing the expression of the peptide (for example by means of low molecular weight inhibitors such as for example siRNA or shRNA), or by directly inhibiting the activity of the protein by administering a NeuromedinU inhibitor or an antibody that has specific binding affinity for NeuromedinU. In a preferred embodiment of the invention, the invention relates to a low molecular weight inhibitor of NeuromedinU expression, the details of which will be well known to the person skilled in the field of molecular biology, and which include siRNA, shRNA, miRNA, antisense oligonucleotides, and ribozyme molecules. Small inhibitory RNA (siRNA) are small double stranded RNA molecules which induce the degradation of mRNAs. Micro RNA's (miRNAs) are single stranded (˜22nt) non-coding RNAs (ncRNAs) that regulate gene expression at the level of translation. Alternatively, small hairpin RNA (shRNA) molecules are short RNA molecules having a small hairpin loop in their tertiary structure that may be employed to silence genes. The design of miRNA or shRNA molecules capable of silencing NeuromedinU will be apparent to those skilled in the field of miRNA or shRNA molecule design (examples of siRNA molecules capable of inhibiting the expression of human neuromedinU are provided in SEQ ID NO: 3 and 4 below). As an alternative, the level of tumour NeuromedinU expression can be modulated using antisense or ribozyme approaches to inhibit or prevent translation of NeuromedinU mRNA transcripts or triple helix approaches to inhibit transcription of the NeuromedinU gene. Antisense approaches involve the design of oligonucleotides (either DNA or RNA) that are complementary to NeuromedinU mRNA. The antisense oligonucleotides will bind to the complementary mRNA transcripts and prevent translation. Ribozyme molecules designed to catalytically cleave NeuromedinU mRNA transcripts can also be used to prevent translation and expression of NeuromedinU. (See, e.g., PCT International Publication W090/11364, published Oct. 4, 1990; Sarver et al. , 1990, Science 247: 1222-1225).

In one embodiment of the invention, the NeuromedinU inhibitor is a NeuromedinU antagonist. One example of a NeuromedinU antagonist is an anti-NeuromedinU antibody (i.e. an antibody which specifically binds to human NeuromedinU peptide or precursor protein). Examples of such antibodies are provided in: http://www.biocompare.com/pfu/110447/soids/325710/Antibodies/neuromedin_U.

NeuromedinU-specific antibodies may be produced using methods which are generally known in the art. In particular, purified NeuromedinU may be used to produce antibodies or to screen libraries of pharmaceutical agents to identify those which specifically bind NeuromedinU. Antibodies to NeuromedinU may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302). For the production of antibodies, various hosts including goats, rabbits, rats, mice, camels, dromedaries, llamas, humans, and others may be immunized by injection with NeuromedinU or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol.

It is preferred that the oligopeptides, peptides, or fragments used to induce antibodies to NeuromedinU have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of NeuromedinU amino acids may be fused with those of another protein, such as KLH, and antibodies to the chimeric molecule may be produced. Monoclonal antibodies to NeuromedinU may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; and Cole, S. P. et al. (1984) Mol. Cell. Biol. 62:109-120.)

In addition, techniques developed for the production of “chimeric antibodies”, such as the splicing of mouse antibody genes to human antibody genes to obtain a molecule with appropriate antigen specificity and biological activity, can be used (see, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454). Alternatively, techniques described for the production of single chain antibodies may be adapted, using methods known in the art, to produce NeuromedinU-specific single chain antibodies. Antibodies with related specificity, but of distinct idiotypic composition, may be generated by chain shuffling from random combinatorial immunoglobulin libraries (see, e.g., Burton, D. R. (1991) Proc. Natl. Acad. Sci. USA 88:10134-10137.). Antibodies may also be produced by inducing in vivo production in the lymphocyte population or by screening immunoglobulin libraries or panels of highly specific binding reagents as disclosed in the literature (see, e.g., Orlandi, R. et al. (1989) Proc. Natl. Acad. Sci. USA 86:3833-3837; Winter, G. et al. (1991) Nature 349:293-299).

Antibody fragments which contain specific binding sites for NeuromedinU may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity (see, e.g., Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between NeuromedinU and its specific antibody. A two-site, monoclonal-based immunoassay utilizing monoclonal antibodies reactive to two non-interfering NeuromedinU epitopes is generally used, but a competitive binding assay may also be employed. Various methods such as Scatchard analysis in conjunction with radioimmunoassay techniques may be used to assess the affinity of antibodies for NeuromedinU. Affinity is expressed as an association constant, Kₐ, which is defined as the molar concentration of NeuromedinU-antibody complex divided by the molar concentrations of free antigen and free antibody under equilibrium conditions. The Kₐ determined for a preparation of polyclonal antibodies, which are heterogeneous in their affinities for multiple NeuromedinU epitopes, represents the average affinity, or avidity, of the antibodies for NeuromedinU. The Kₐ determined for a preparation of monoclonal antibodies, which are monospecific for a particular NeuromedinU epitope, represents a true measure of affinity. High-affinity antibody preparations with Kₐ ranging from about 10⁹ to 10¹² L/mole are preferred for use in immunoassays in which the NeuromedinU-antibody complex must withstand rigorous manipulations.

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of NeuromedinU-antibody complexes. Procedures for evaluating antibody specificity, titer, and avidity, and guidelines for antibody quality and usage in various applications, are generally available.

The invention provides a method of treating a cancer, especially a HER2-positive cancer, or increasing the sensitivity (or reducing the resistance) of a cancer, typically a HER2-positive cancer, to a HER2 targeting drug or agent, comprising a step of administering to the individual a therapeutically effective amount of a NeuromedinU inhibitor optionally in conjunction with administration of a therapeutically effective amount of theHER2 targeting drug. The NeuromedinU inhibitor may be administered together with the chemotherapeutic agent (for example at the same time or as part of a single dose), or it may be administered in advance of or after administration of the chemotherapeutic agent. In this context, the term “therapeutically effective amount” typically refers to an amount of NeuromedinU inhibitor which increases the sensitivity (or decreases the resistance) of the tumour cell to the HER2 targeting drug compared to a tumour cell which has not be treated with a NeuromedinU inhibitor.

In this specification, the term “treating” refers to administering a NeuromedinU inhibitor, optionally in combination with a HER2 targeted drug, to an individual that has a HER2-positive cancer, typically a solid tumour cancer, with the purpose to cure, heal, prevent, alleviate, relieve, alter, remedy, ameliorate, or improve the cancer or symptoms of the cancer. When the term is applied to the use of a NeuromedinU inhibitor and a HER2 targeted drug, the respective active agents may be administered together, or separately, and may be administered at the same time or at different times. In one embodiment, the patient may be treated to a course of one active agent, which is then followed by treatment with a course of the second active agent. The term “therapeutically effective amount” refers to the amount of the NeuromedinU inhibitor or chemotherapeutic agent/therapy that is required to confer the intended therapeutic effect in the individual, which amount will vary depending on the type of inhibitor, route of administration, status of cancer, and possible inclusion of other therapeutics or excipients.

The methods of the invention apply to HER2-positive tumours, especially to solid tumour cancers (solid tumours), which are cancers of organs and tissue (as opposed to haematological malignancies), and ideally epithelial cancers. Examples of solid tumour cancers include pancreatic cancer, bladder cancer, prostate cancer, ovarian cancer, colorectal cancer (CRC), breast cancer, renal cancer, lung cancer, hepatocellular cancer, cervical cancer, gastric cancer, esophageal cancer, head and neck cancer, melanoma, neuroendocrine cancer. Suitably, the solid tumour cancer suitable for treatment and prognosis according to the methods of the invention are selected from CRC, breast and prostate cancer. In a preferred embodiment of the invention, the invention relates to treatment and prognosis of breast cancer, and in particular, HER2-positive breast cancer. In another aspect, the methods of the invention apply to treatment and prognosis of outcome of haematological malignancies, including for example multiple myeloma, T-cell lymphoma, B-cell lymphoma, Hodgkins disease, non-Hodgkins lymphoma, acute myeloid leukemia, and chronic myelogenous leukemia.

In this specification, the term “increasing the responsiveness” of a HER2 targeting drug should be understood to mean reducing the resistance of a HER2-positive cancer cell to the effect of a HER2 targeting drug.

Various delivery systems are known and can be used to administer a therapeutic of the invention. Methods of introduction include but are not limited to intradermal, intramuscular, intraperitoneal, intravenous, subcutaneous, intranasal, epidural, intranasal, intracerebral, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the compositions of the invention locally to the area in need of treatment; this may be achieved, for example and not by way of limitation, by topical application, by injection, by means of a catheter, by means of a suppository, or by means of an implant, said implant being of a porous, non-porous, or gelatinous material, including membranes, such as sialastic membranes, or fibers.

The present invention also provides pharmaceutical compositions. Such compositions comprise a therapeutically effective amount of the therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. In this specification, the term “therapeutically effective amount” should be taken to mean an amount of therapeutic which results in a clinically significant inhibition, amelioration or reversal of development or occurrence of seizures or, in the case of treatment of stroke, clinically significant inhibition, amelioration or reversal of development of the effects of stroke.

The term “carrier” refers to a diluent, adjuvant, excipient, or vehicle with which the Therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the, site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The screening assays of the invention may be performed on any mammalian cell that expresses NeuromedinU (and are ideally HER2-positive), for example SKBR3 and HCC1954 cells (the details of which are provided below). The cells are typically incubated with a test agent, and the expression of NeuromedinU is monitored to detect changes in expression due to the test agent. In one embodiment, the cells are pre-treated or co-treated with a HER2 targeted drug.

Embodiments of the invention also provide for systems (and computer readable media for causing computer systems) to perform: a method for detecting/identifying/predicting responsiveness/resistance of a HER2-positive patient to HER2 targeted agents; a method of identifying/detecting/predicting aggressiveness of a HER2-positive cancer; a method of identifying/detecting/predicting metastatic potential of a HER2-positive cancer; or a method of predicting poor outcome of a patient with a HER2-positive cancer.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media can be any available tangible media that can be accessed by a computer. Computer readable storage media includes volatile and non-volatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable storage media may define instructions, for example, as part of one or more programs, that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable storage media on which such instructions are embodied may reside on one or more of the components of either of a system, or a computer readable storage medium described herein, may be distributed across one or more of such components.

The computer-readable storage media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include at minimum a determination system, a storage device, a comparison module, and a display module. The functional modules can be executed on one, or multiple, computers, or by using one, or multiple, computer networks. The determination system has computer executable instructions to provide e.g., sequence information in computer readable form.

The determination system can comprise any system for detecting the level of NeuromedinU in a biological sample from the patient. Standard procedures may be used.

The information determined in the determination system can be read by the storage device. As used herein the “storage device” is intended to include any suitable computing or processing apparatus or other device configured or adapted for storing data or information. Examples of an electronic apparatus suitable for use with the present invention include a stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon nucleic acid or protein/peptide adundance information. Such information may be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising information relating to peptide or nucleic acid abundance information.

In one embodiment the reference data stored in the storage device to be read by the comparison module is compared.

The “comparison module” can use a variety of available software programs and formats for the comparison operative to compare abundance levels for NeuromedinU to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related to sample information.

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content based in part on the comparison result that may be stored and output as requested by a user using a display module.

In one embodiment of the invention, the content based on the comparison result is displayed on a computer monitor. In one embodiment of the invention, the content based on the comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

The methods described herein therefore provide for systems (and computer readable media for causing computer systems) to perform methods as described in the Statements of Invention above, for example (a) methods of identifying the responsiveness of a HER2-positive cancer in a patient to a HER2 targeted drug (b) a method of identifying an aggressive HER2-positive cancer in a patient (c) a method of assessing the metastatic potential of a HER2-positive cancer in a patient (d) method of prediction of poor outcome in a patient having a HER2-positive cancer (e) a method for identifying an agent capable of treating or preventing HER2-positive cancer in a patient, and (f) method for identifying an agent capable of increasing the responsiveness of a HER2-positive cancer in a patient to a HER2 targeted drug.

Systems and computer readable media described herein are merely illustrative embodiments of the invention for performing methods of diagnosis in an individual, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### Materials and Methods

### Cell Culture and Treatments

SKBR3, HCC1954, MDA-MB-361, T47D cells, obtained from ATCC, were cultured in RPMI-1640 (Sigma-Aldrich) with 10% FCS (PAA) and 1% L-glutamine. Trastuzumab-conditioned (resistant) SKBR3 (SKBR3-TR) cells were established by continuous exposure to 1.4 μM Trastuzumab for 9 months. Lapatinib-resistant SKBR3 and HCC1954 cells (SKBR3-LR, HCC1954-LR) were established by continuously exposing cells to Lapatinib, starting with 5 nM and incrementally increasing to 250 nM over 6 months. Neratinib-resistant SKBR3 and HCC1954 cells (SKBR3-NR, HCC1954-NR) resulted from continuously exposing cells to Neratinib, starting with 10 nM and incrementally increasing to 250 nM over 6 months.

To assess if functional NmU receptors are expressed on cells, SKBR3 and HCC1954, (1×10⁶ cells) were seeded in 25 cm² flask, allowed attach overnight and subsequently treated with 1 μM of NmU-25 (Bachem, Switzerland) for 24-48 hours.

### Short-Term Drug Exposure Assays

SKBR3 and HCC1954 cells were seeded (5×10⁵ cells, 25 cm² flasks) and allowed to grow to 80% confluency before being exposed, for 48 hours, to Lapatinib (1 μM), Trastuzumab (12.5 μg/ml), Neratinib (0.5 μM), or Afatinib (0.5 μM).

RNA Isolation from Conditioned Medium

Conditioned medium (CM) was collected, centrifuged and filtered, as previously described (1-2), prior to NmU mRNA and protein analysis.

### NmU Knock-Down and Over-Expression

Two siRNAs, designated NmU-1 (SEQ ID NO: 3; 5′-AAAGGTTCTTATTTCATTA-3′) and NmU-2 (SEQ ID NO: 4; 5′-AGATGATACTTGTTCGTCT-3′) (s225456 and s21351, respectively, Ambion, UK), (30 nM) were used to target NmU. Scrambled siRNA (SCR) (AM4611, Ambion, UK) was used as negative control. Transient transfections were harvested 72 hours post-transfection for RNA and protein extraction. NmU full-length cDNA was sub-cloned from pOTB7 (clone ID3502168, Open Biosystems, Ireland) by PCR into pcDNA 3.1(+) zeo-plasmid vector (Invitrogen, Ireland). The construct was verified by DNA sequencing. Mock controls used were of pcDNA3.1(+) plasmid lacking NmU cDNA. Lipofectamine 2000 (Invitrogen, Ireland) was used for transfection following the manufacturer's instructions. Stable transfectants were established by selecting with zeocin (50 μg/ml and 300 μg/ml for HCC1954 and SKBR3 transfected cells, respectively; based on initial assessment of the toxicity of zeocin on these cells) (Invitrogen, Ireland).

### Proliferation Assays

Cells with acquired-resistance compared to aged-parent cells and NmU cDNA-versus mock-transfected cells (HCC1954 variants, 2×10³ cells/well; SKBR3 variants, 5×10³ cells/well) were seeded for 24 hours prior to drug additions. Subsequently, Lapatinib (0-1.1 μM for SKBR3; 0-6 μM for SKBR3-LR; 0-500 nM for both SKBR3-mock and SKBR3-NmU; 0-1.1 μM for HCC1954; 0-6 μM for HCC1954-LR; 0-800 nM for HCC1954-mock; 0-15 μM for HCC1954-NmU), Neratinib (2-40 nM for both SKBR3-mock and SKBR3-NmU; 0-750 nM for HCC1954-mock; 0 nM-1.2 μM for HCC1954-NmU), Trastuzumab (0-500 μg/ml for both HCC1954-mock and HCC1954-NmU) or Afatinib (0-32 nM for SKBR3-mock; 0-100 nM for SKBR3-NmU; 0-50 nM for HCC1954-mock; 0-185 nM for HCC1954-NmU) was added to the cells. Five days later, acid phosphatase assay was performed as previously detailed (3).

Assessing Effects of NmU siRNAs with HER-Targeted Drugs

To investigate if knock-down of NmU affected response to HER-targeting, cell lines with acquired Lapatinib-resistance (SKBR3-LR, HCC1954-LR) and innately unresponsive (MDA-MB-361, T47D) were assessed. Forty-eight hours following transfection with NmU-1 siRNA, NmU-2 siRNA or SCR sequences, cells were exposed to their approximate IC₅₀ concentrations of Lapatinib (3 μM, SKBR3-LR; 5 μM, HCC1954-LR; 1 μM, MDA-MB-361; 5 μM, T47D), as these IC₅₀ values had previously been determined. As in previous studies, a fixed concentration of Trastuzumab (15 μg/ml) was assessed for all 4 cell line variants. Similarly, fixed concentrations of Neratinib (1 μM) and Afatinib (0.5 μM) were used. These were cultured for 72 hours and subsequently assessed using acid phosphate analysis.

qPCR

Total RNA was isolated from cell lines and CM using TriReagent (Sigma-Aldrich). In order to remove any potential contaminating genomic DNA, RNA was treated with DNase enzymes and minus reverse transcriptase enzyme controls verified no DNA/pseudogene contamination of starting material. cDNA was prepared from 500ng cell-derived and 4 μl CM-derived total RNA, respectively. NmU (Hs00183624_ml, ABI, UK) was quantified using the threshold cycle (C_{T}) adjusting to the levels of β-actin (4352933E, ABI, UK), established as not differing significantly in expression levels between cell populations being assessed and so suitable as endogenous control.

### Immunoblotting and Enzyme-Linked Immunosorbent Assay

Total cellular proteins (30-40 μg, depending on the specific protein's abundance; but constant for any given protein) were resolved on 6-10% SDS-PAGE and transferred to PVDF membranes (Millipore, Ireland). Primary antibodies used included EGFR (Neomarker); HER2 (Calbiochem); Akt, p-Akt (Cell Signalling); NmU-R1 (Sigma-Aldrich); NmU-R2 (LifeSpan Biosciences); β-actin (Sigma-Aldrich). Membranes were incubated with appropriate horseradish peroxidase-conjugated secondary antibodies (Cell Signalling) and proteins were visualized by chemiluminescence (Millipore). Detection was performed with a Chemidoc exposure system (Bio-Rad Laboratories). ELISA kit for NmU (Peninsula Laboratories, US), HER2 (Calbiochem, US), p-HER2, EGFR, p-EGFR (R&D Systems, US) were used according to the manufacturer's instructions.

### Wound-Healing Assay

HCC1954-Ag, HCC1954-LR and associated SCR- or siRNA-transfected cell variants (5×10⁵ cells/well) and SKBR3-Ag, SKBR3-LR associated SCR- or siRNA-transfected cell variants (1×10⁶ cells/well) were seeded on 6-well plates and cultured for 48 hours to confluency. Monolayer was scratched with a pipette tip and the resulting wounded areas were monitored by phase contrast microscopy and determined using NIH Image J software.

### Migration and Invasion Assay

Migration assays were performed using 8 μm pore size 24-well transwell chambers (BD Biosciences, Oxford, UK). For invasion assessment, the inserts were pre-coated with ECM (Sigma-Aldrich). Acquired resistance, SCR/siRNA, and cDNA over-expressing variants compared to controls (HCC1954 variants, 1×10⁵/insert; SKBR3 variants, 1×10⁶/insert) were seeded in the upper compartment and allowed to migrate for 48 and 72 hours, respectively. Cells in the upper chamber were removed, migrated/invaded cells were stained with crystal violet, staining was solubilised in 10% acetic acid, and read at 595 nm.

### Anoikis Assay

Cells with acquired resistance compared to aged-parent cells; siRNA- or SCR-transfected cells; and NmU cDNA-versus mock-transfected cells (HCC1954 variants, 1×10⁵ cells/well; SKBR3 variants, 1×10⁴ cells/well) were seeded onto a 24-well plates coated with Poly(hydroxyethyl methacrylic) acid (Sigma-Aldrich) or 95% ethanol and cultured for 24 and 48 hours, respectively. Alamar blue dye (100 μl ; Serotec, UK) was added/well and absorbance read at 570 nm; reference wavelength, 600 nm.

### Assessing Potential Clinical Relevance of NmU in Breast Cancer

NmU expression was evaluated in publically-available microarray data from 21 datasets representing 3,489 breast tumours, including the luminal A (n=1,521), luminal B (n=676), HER2 (n=476) and basal (n=454) molecular subtypes. Gene expression datasets were downloaded from Gene Expression Omnibus (http://www.ncbi.nlm.nih.gov/geo/) or authors' websites in the form of raw data files where possible. Table 1 below provides a list of the datasets used. In total 3,489 specimens across 10 different platforms were analysed (30 specimens were removed as they lacked clinical information). Where raw data was unavailable, the normalised data—as published by the original study—was used. In the case of the Affymetrix datasets (.cel files), gene expression values were called using the robust multichip average method (4) and data were quantile normalised using the Bioconductor package, affy (www.bioconductor.org). For the dual-channel platforms, data were loess normalised using the Bioconductor package limma. The R package genefu was used to classify the specimens into the luminal A (n=1,521), luminal B (n=676), HER2 (n=476) and basal (n=454) molecular subtypes using the ssp2003 classifier. 362 specimens did not fall into any of these groups. The Entrez gene ID (10874) corresponding to the array probe targeting NmU was obtained from the Gene database at NCBI (www.ncbi.nlm.nih.gov/gene/). All calculations were carried out in the R statistical environment (http://cran.r-project.org/).

TABLE 1
Twenty-one breast cancer microarray datasets collated and assessed for NmU in
association with patients outcome.

| GEO | | Specimen | | |
|---|---|---|---|---|
| Accession | Availability | Number | Platform Type | Reference |
| GSE7849 | Processed only | 78 | Affymetrix Human Genome U95 | (5) |
| | | | Version 2 Array | |
| GSE3143 | Raw CEL files | 158 | Affymetrix Human Genome U95 | (6) |
| | | | Version 2 Array | |
| GSE10510 | Raw data | 152 | DKFZ Division of Molecular | (7) |
| | available | | Genome Analysis Human Operon | |
| | | | 4.0 oligo Array 35k | |
| NA | Processed only | 295 | Agilent | (8) |
| NA | Processed only | 118 | Affymetrix U133AA of Av2 | (9) |
| GSE9893 | Raw data | 155 | MLRG Human 21K V12.0 | (10) |
| | available | | | |
| GSE7390 | Raw CEL files | 198 | Affymetrix U133A | (11) |
| GSE16391 | Raw CEL files | 48 | Affymetrix U133 Plus 2.0 | (12) |
| GSE1992 | Processed only | 99 | Agilent | (13) |
| GSE4922 | Raw CEL files | 249 | Affymetrix U133A/B | (14) |
| NA | Processed only | 69 | Agilent 44K oligo array | (15) |
| GSE9195 | Raw CEL files | 77 | Affymetrix U133 Plus 2.0 | (16) |
| GSE6532 | Raw CEL files | 414 | Affymetrix U133A/B and plus2 | (16) |
| GSE1378, | Processed only | 60 | Custom 22K oligo array | (17) |
| GSE 1379 | | | | |
| GSE3494 | Raw CEL files | 251 | Affymetrix U133A/B | (18) |
| GSE1456 | Raw CEL files | 159 | Affymetrix U133A/B | (19) |
| GSE21653 | Raw CEL files | 266 | Affymetrix U133 Plus 2.0 | (20) |
| GSE17907 | Raw CEL files | 51 | Affymetrix U133 Plus 2.0 | (20) |
| GSE11121 | Raw CEL files | 200 | Affymetrix U133A | (21) |
| GSE2034 | Raw CEL files | 286 | Affymetrix U133A | (22) |
| GSE12093 | Raw CEL files | 136 | Affymetrix U133A | (23) |

### Statistical Analysis

Statistical analysis on cell line- and CM-derived data was performed in Excel. P values were generated using Student's T-tests, with p<0.05 considered as statistically significant. GraphPad Prism 5.0 was used for graph generation. (Graph Pad Software Inc, La Jolla, USA).

Relapse-free survival (RFS) of untreated patients was considered the survival end-point. When RFS information was unavailable, distant metastasis-free survival (DMFS) data was used and, if neither RFS nor DMFS were available, overall survival (OS) was used. Median expression was used to determine high and low expression groups within each of the 21 individual datasets. Once a specimen was assigned to a particular group, the 21 datasets were combined and a global survival analysis was performed. Each dataset was considered separately when determining if a specimen belongs to the high or low expression groups, as the expression of mRNAs (including NmU) varies across the different experiments/platforms. The survival curves were based on Kaplan-Meier estimates and Cox proportional hazards regression was used to estimate proportional hazards for the NmU gene expression and other clinicopathological variables, in both univariate and multivariate models. R package survival was used to calculate and plot the Kaplan-Meier survival curve.

### Results

Intracellular and Extracellular NmU mRNA Levels are Associated with Acquired Resistance to Lapatinib.

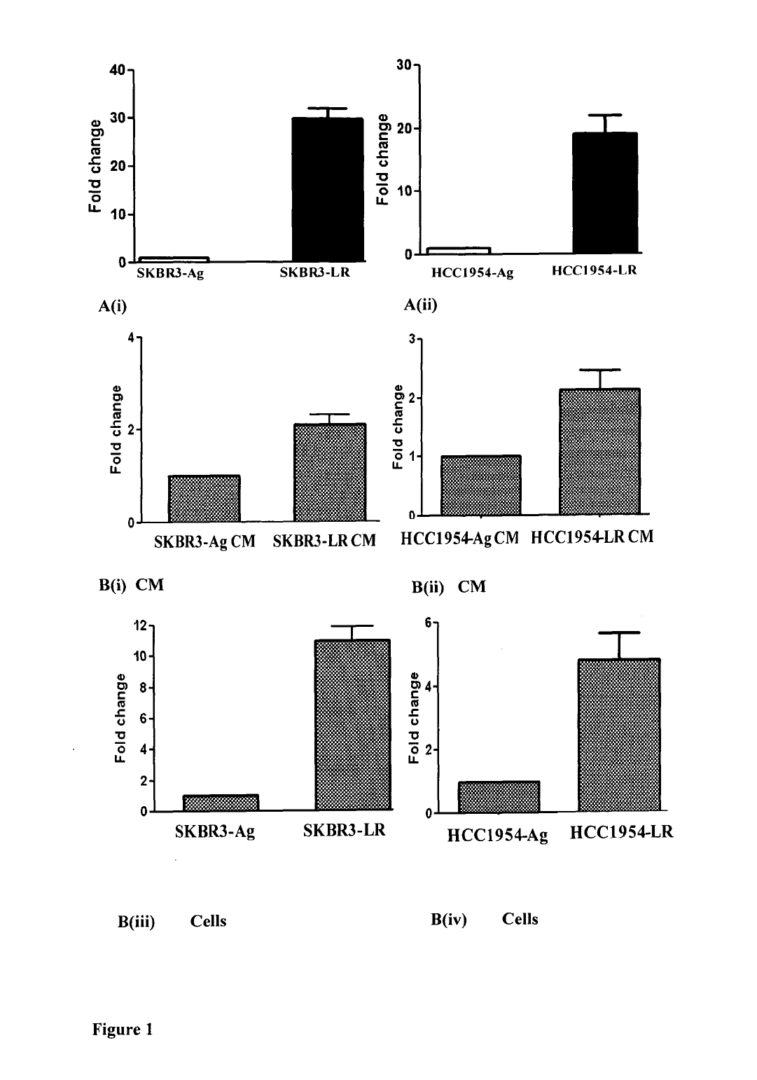

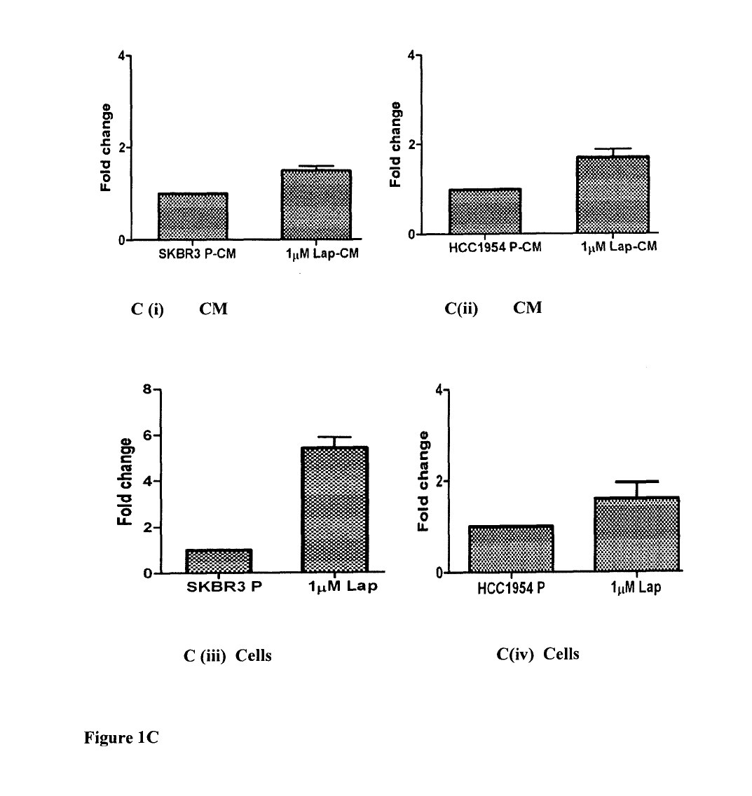

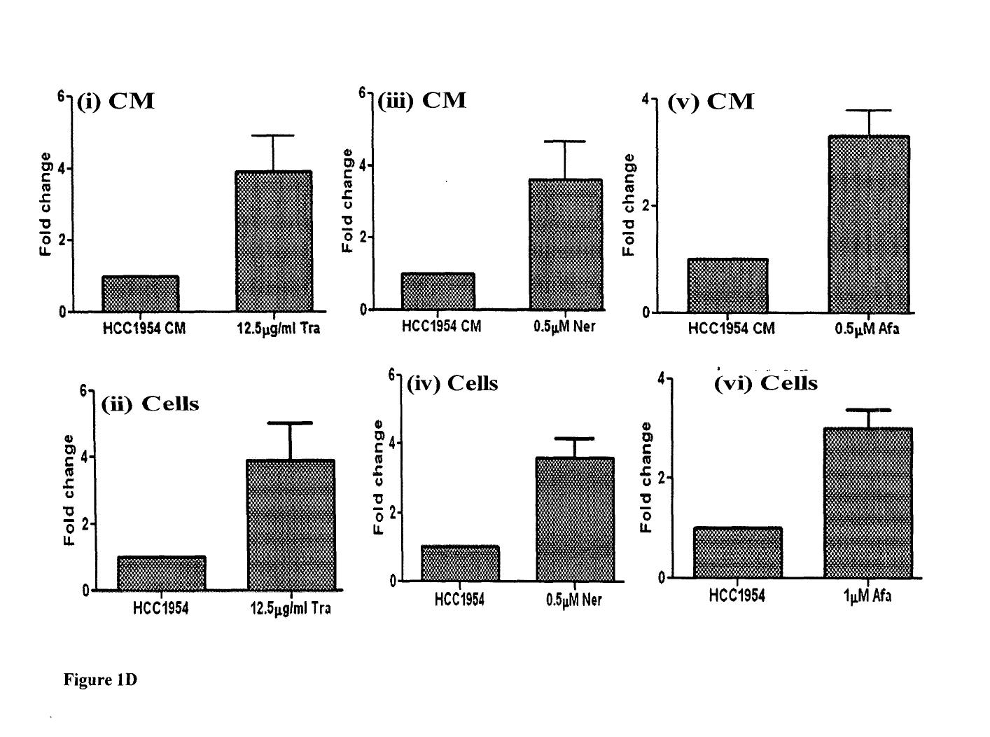

In the efforts to identify extracellular—as well as intracellular—mRNAs that may be associated with resistance to HER-targeted agents, the initial analysis included HER2 over-expressing cell line models, SKBR3 and HCC1954, that were conditioned with Lapatinib over an approximate 6-month time period to resulting in cell populations that were termed Lapatinib-resistant (LR) compared to their aged-parent populations. Comparing the concentration of Lapatinib that inhibits 50% of proliferation (IC₅₀) for SKBR3 that had acquired resistance (SKBR3-LR) (IC₅₀=3 μM) in relation to SKBR3 aged cells (IC₅₀=0.09 μM) as controls (and so termed SKBR3-Ag), a 29.8±2.2 fold resistance to Lapatinib was observed. For HCC1954 cells, a similar trend was observed with a 19.1±2.8 fold resistance to Lapatinib in HCC1954-LR (IC₅₀=5 μM) compared to its age-matched population, HCC1954-Ag (IC₅₀=0.3 μM) (FIG. 1A).

Evaluating mRNAs in medium conditioned by SKBR3-LR and HCC1954-LR cells, compared to conditioned medium (CM) from their age-matched control cell lines, showed significantly higher levels of extracellular NmU to be associated with Lapatinib resistance. This observation was validated by qPCR in CM from these cells (FIG. 1B(i) & (ii)). The trend of increased NmU mRNA levels observed in CM from the resistant compared to the sensitive cell lines was subsequently found to reflected that in the corresponding cells (FIG. 1B(iii) & (iv)).

Investigating if the induced expression of NmU may be an early response to drug exposure, cells were treated with Lapatinib (1 μM) for 48 hours and it was found that the levels of NmU mRNA detectable in CM from the Lapatinib-exposed SKBR3 and HCC1954 cells (FIG. 1C(i) & (ii)) were significantly higher than in the corresponding untreated control CM, even after this relatively short-term exposure to drug. As expected, a similar trend was found in the corresponding cells (FIG. 1C(iii) & (iv)). Considering a broader range of HER-targeted drugs, treating cells (HCC1954 as example; data for SKBR3 not shown) for 48 hours with Trastuzumab (12.5 μg/ml) resulted in induced NmU mRNA levels in CM (3.9±1 fold; p=0.04) and corresponding cells (3.9±1.15 fold; p=0.06) (FIG. 1D(i) & (ii)). In relation to Neratinib (0.5 μM), the resulting induced NmU mRNA was 3.6±1 fold (p=0.07) and 3.6±0.54 fold (p=0.009) and CM and cells, respectively (FIG. 1D(iii) & (iv)); and for Afatinib (0.5 μM), induced NmU mRNA was 3.3±0.49 fold (p=0.01) in CM and 3±0.38 fold (p=0.006) in cells (FIG. 1D(v) & (vi)).

Induced NmU Protein Expression Occurs in Cells with Acquired Resistance to Other HER-Targeted Agents and is not Restricted to Lapatinib.

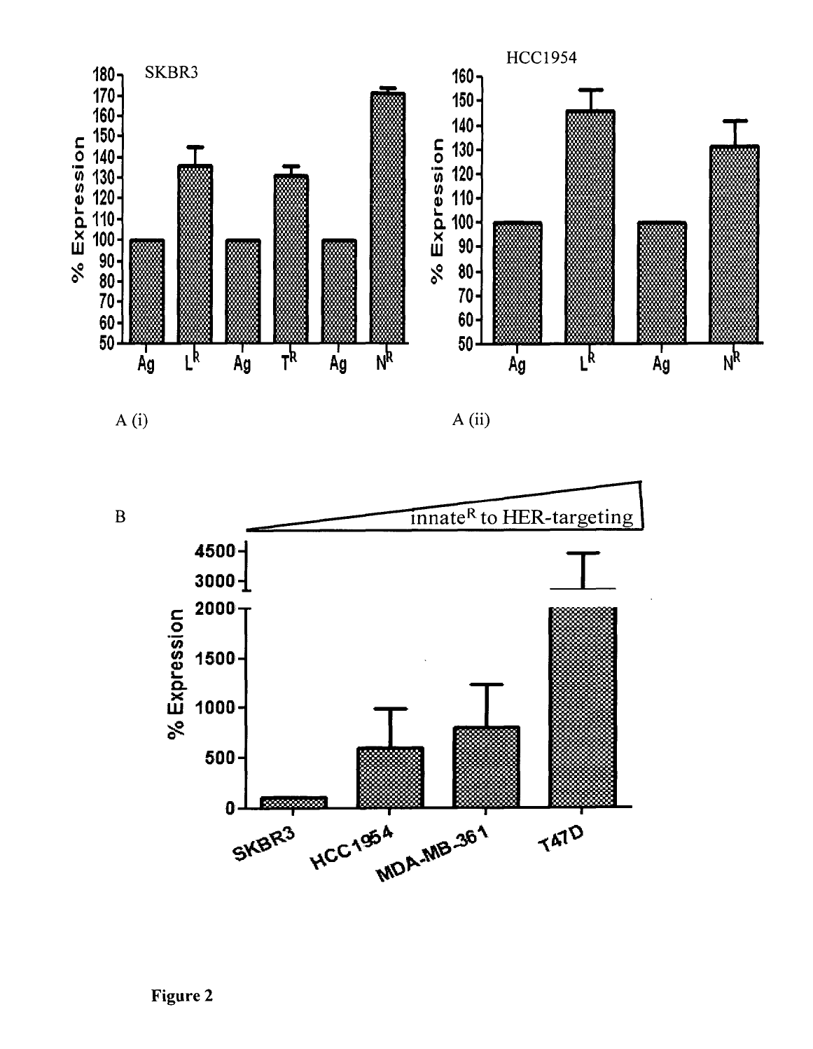

The changes at the mRNA level were tested in acquired-resistant cells translated to NmU protein. In agreement with the mRNA observations, NmU protein levels were significantly higher in Lapatinib-conditioned cells (SKBR3-LR, HCC1954-LR) compared to their aged-matched control cells (SKBR3-Ag, HCC1954-Ag) (FIG. 2A(i) & 2A(ii)). Interestingly, this observation was not specific to Lapatinib, but was also found in relation to other HER-targeted drugs where acquired-resistance populations were available. Specifically, a similar trend (i.e. significantly increased NmU protein levels) was observed when Trastuzumab-resistant (SKBR3-TR (or -TR) and Neratinib-resistant (SKBR3-NR or -NR) cells were compared with their aged-matched control cells (SKBR3-Ag) (FIG. 2A(i)) and the Neratinib-resistant HCC1954 (HCC1954-NR or -NR) cells with their control cells (HCC1954-Ag) cells (FIG. 2A(ii)).

Endogenous NmU Protein Levels are Associated with Innate Sensitivity/Resistance to HER-Targeting Drugs.

SKBR3 cells are sensitive to both Lapatinib and Trastuzumab; HCC1954 cells are sensitive to Lapatinib but resistant to Trastuzumab; conversely, MDA-MB-361 is resistant to Lapatinib, but sensitive to Trastuzumab (22). Although T47D have been previously described as having normal HER2 expression and for also unresponsive to both these drugs, it was elected to include these in the analysis presented herein. As shown in FIG. 2B, endogenous levels of NmU protein correlate with the innate sensitivity versus resistance profile of these 4 cell lines.

NmU Expression is Prognostic for Poor Outcome for Breast Cancer Patients, Particularly Those with HER2 Positive and Luminal A Subtypes.

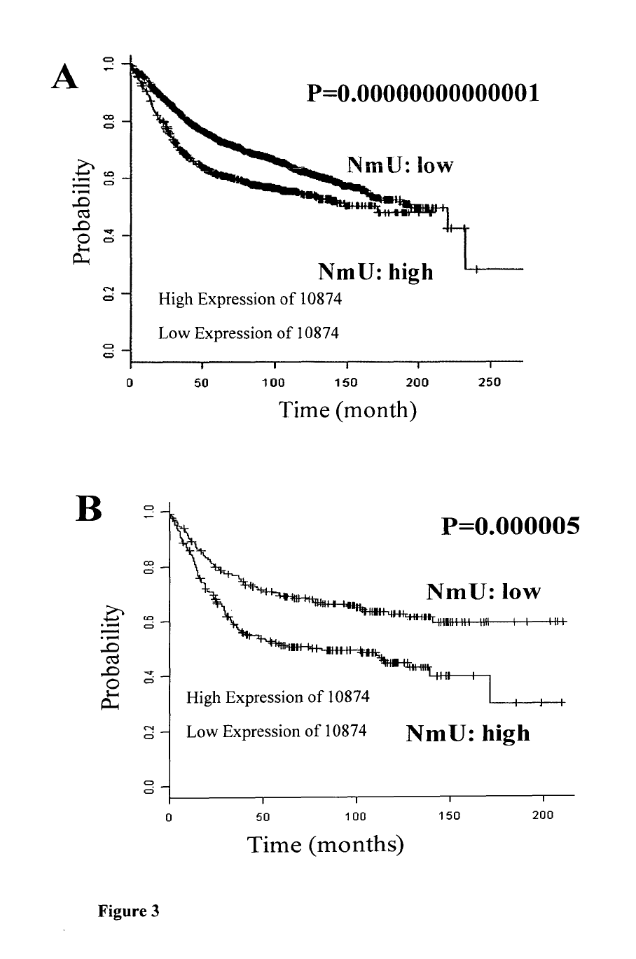

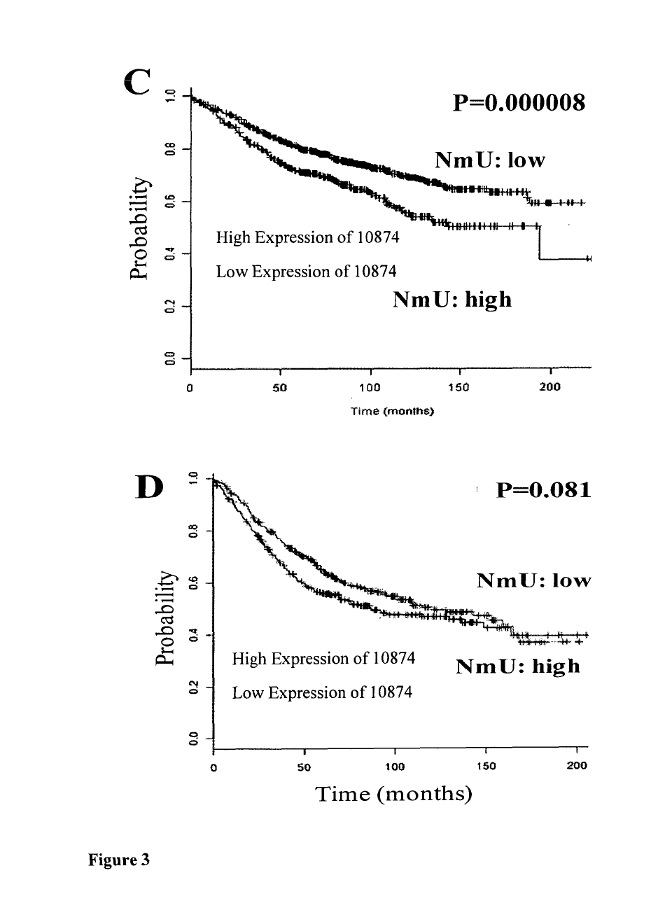

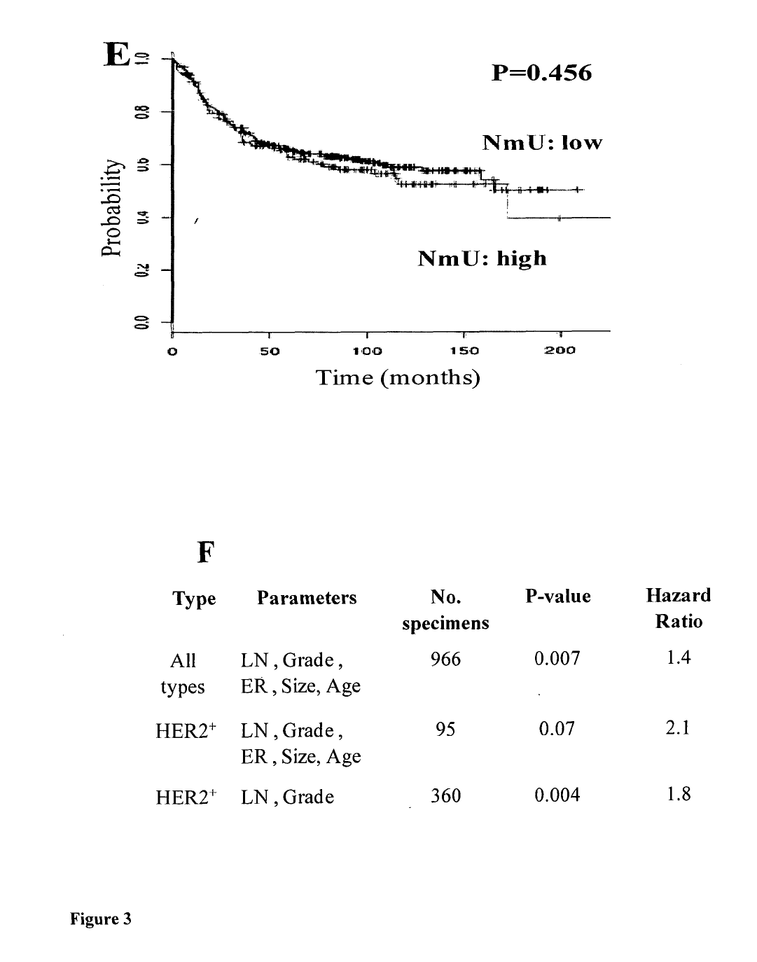

To determine if NmU has relevance in human breast cancer rather than solely a cell line/CM-related observation, microarray data relating to 3,489 breast tumours were collated and mined. Kaplan-Meier estimates of survival (FIG. 3A) indicated high levels of NmU expression to be associated with poor outcome for breast cancer patients (p<1e-14). Considering each of the breast cancer molecular subtypes within this general population of breast tumours, NmU expression was found to be particularly associated with poor outcome for those patients who had HER2-positive tumours (FIG. 3B; p<5e-6) and luminal A tumours (FIG. 3C; p<8e-6). These associations were not significant in patients with luminal B ((FIG. 3D; p=0.081) or basal-like ((FIG. 3E; p=0.456) tumours.

Although complete clinicopathological information was unavailable for all 3,489 patients, multivariate analysis correcting for tumour size, grade, ER status, lymph node status and age of patient, where this information was available, confirmed NmU as an independent prognostic biomarker rather than it being a surrogate for an already established parameter (FIG. 3F). Specifically, considering all tumour types where information on these five parameters was available (n=966), high levels of NmU expression associated with poor outcome (p=0.007; hazard ratio=1.4). Considering the HER2-positive subtype, which is particularly relevant to this study, detailed clinical information was available for only ninety-five patients. Following correction, high levels of NmU in HER2 tumours tended towards significant (p=0.07; hazard ratio=2.1). As information on tumour grade and lymph node status was available for a substantial number of the HER2-positive tumours (n=360 specimens), NmU in this cohort was evaluated and found to be independently associated with poor outcome for HER2-overexpressing patients (p=0.004; hazard ratio=1.8).

### NmU Affects Sensitivity to Lapatinib, Trastuzumab, Neratinib and Afatinib

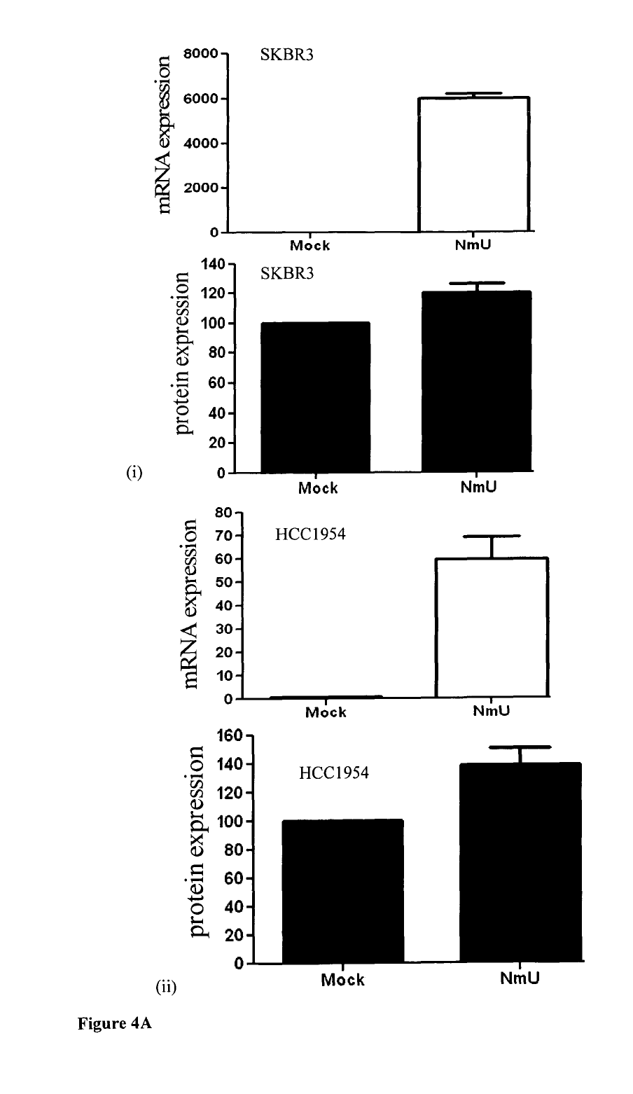

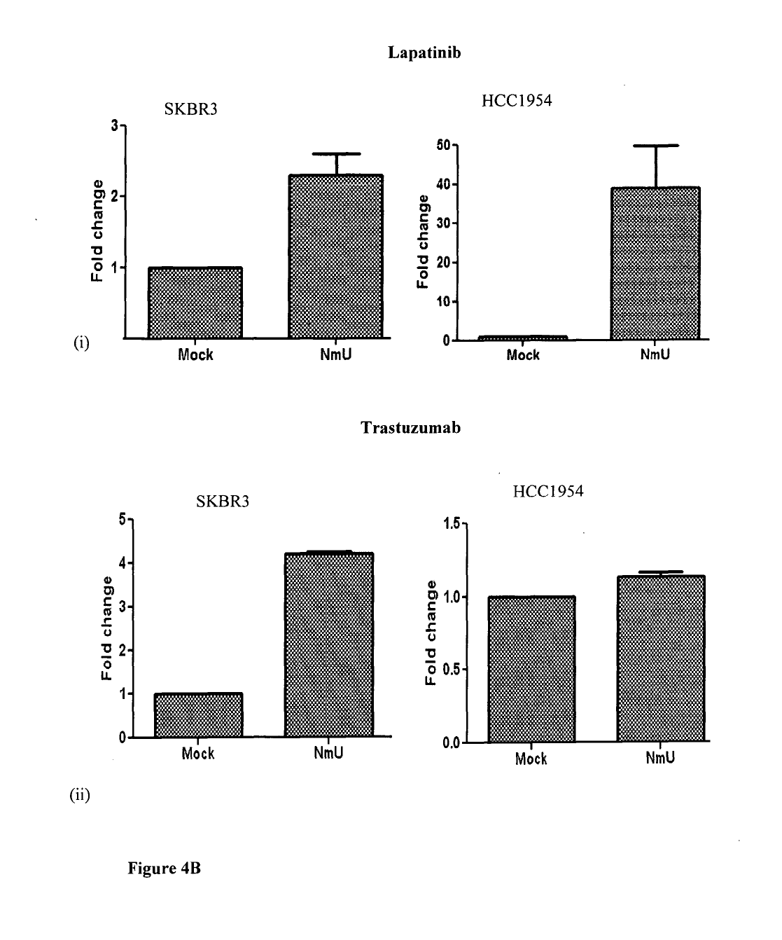

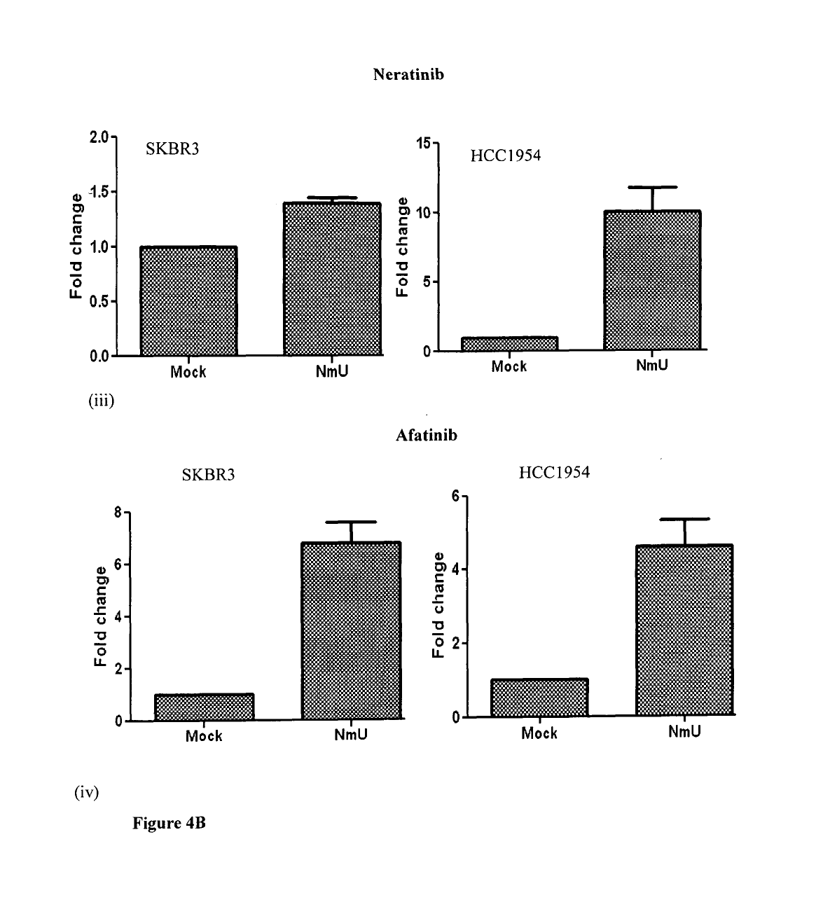

To assess if NmU might be functionally involved in resistance to HER-targeted drugs, we stably transfected human NmU cDNA into SKBR3 and HCC1954 parent cells and established successful over-expression of NmU compared to levels in mock-transfected cells, using qPCR and ELISA (FIG. 4A(i) & (ii)). For all drugs tested i.e. Lapatinib (FIG. 4B(i)), Trastuzumab (FIG. 4B(ii)), Neratinib (FIG. 4B(iii)) and Afatinib (FIG. 4B(iv)), the anti-proliferative effects resulting were significantly compromised in the NmU-transfected cells compared to the mock-transfected cells. The exception to this being the response of HCC1954-NmU compared to HCC1954-mock cells to Trastuzumab (FIG. 4B(ii)).

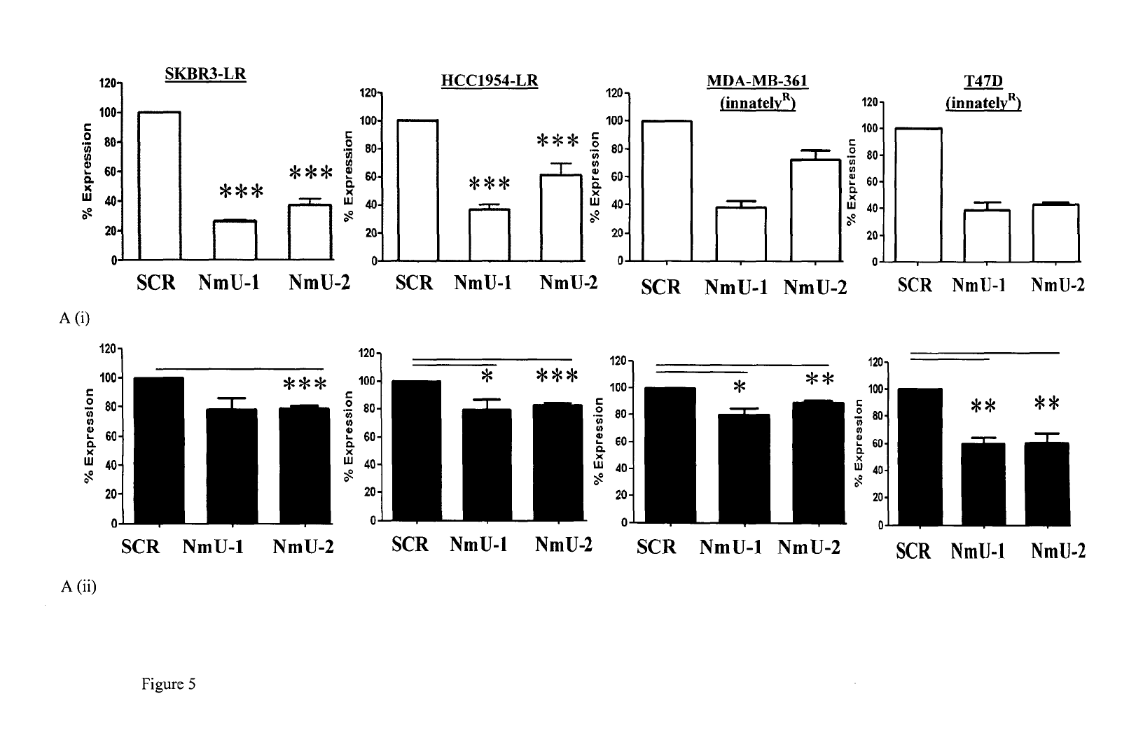

To further explore a functional role for NmU in resistance to HER-targeted drugs, NmU was subsequently knocked-down in both acquired resistant cell lines (namely SKBR3-LR and HCC1954-LR) and innately resistant/unresponsive cells (MDA-MB-361, T47D). Again, qPCR and ELISAs established significant knock-down of NmU mRNA as shown in FIG. 5A(i) and protein FIG. 5A(ii), respectively, compared to levels in scrambled (SCR) control cells. In relation to affects on response to drug, while some variation was observed between cell lines and siRNAs, NmU knock-down was found to increase the inhibition of proliferation achieved in response to Lapatinib (IC₅₀ concentration) by a further 12-49% (FIG. 5B). For Trastuzumab, NmU knock-down added a further 14-50% inhibition of growth, with corresponding values of 19-58% for Neratinib, and 18-47% for Afatinib (FIG. 5B).

### Proposed Mechanism of Action

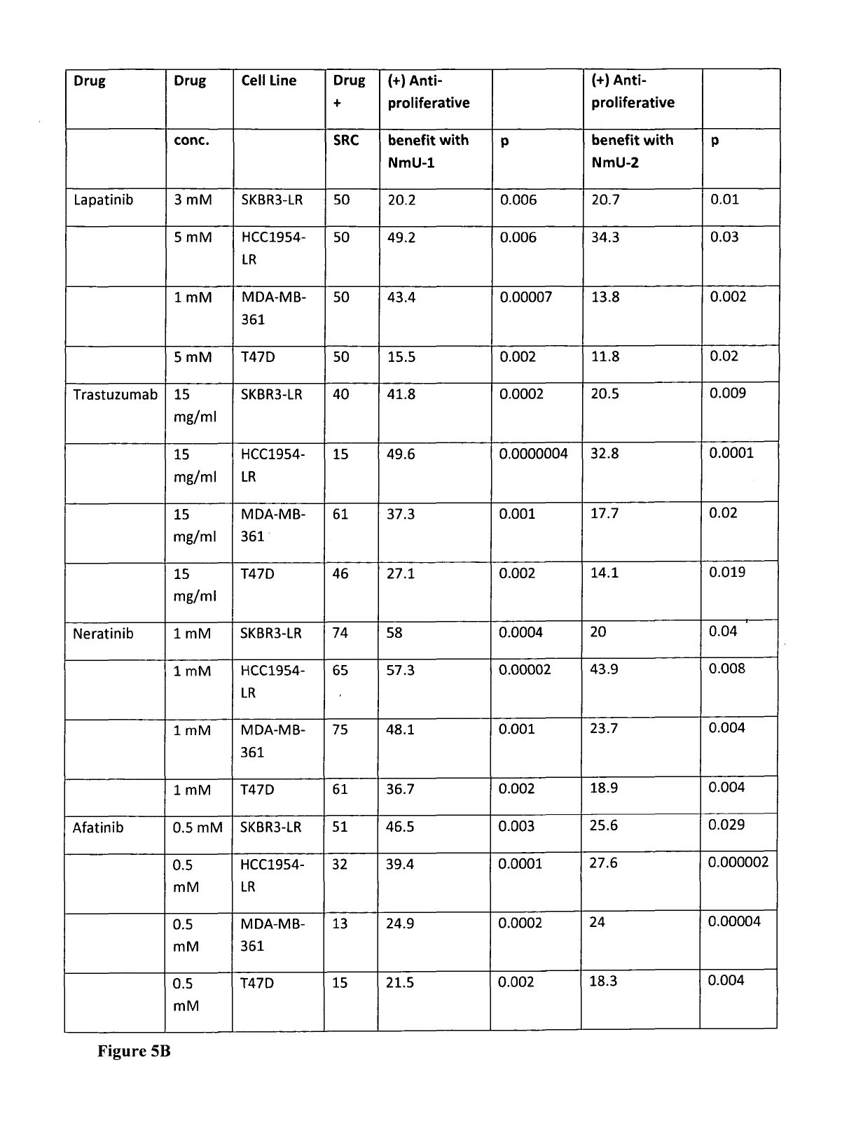

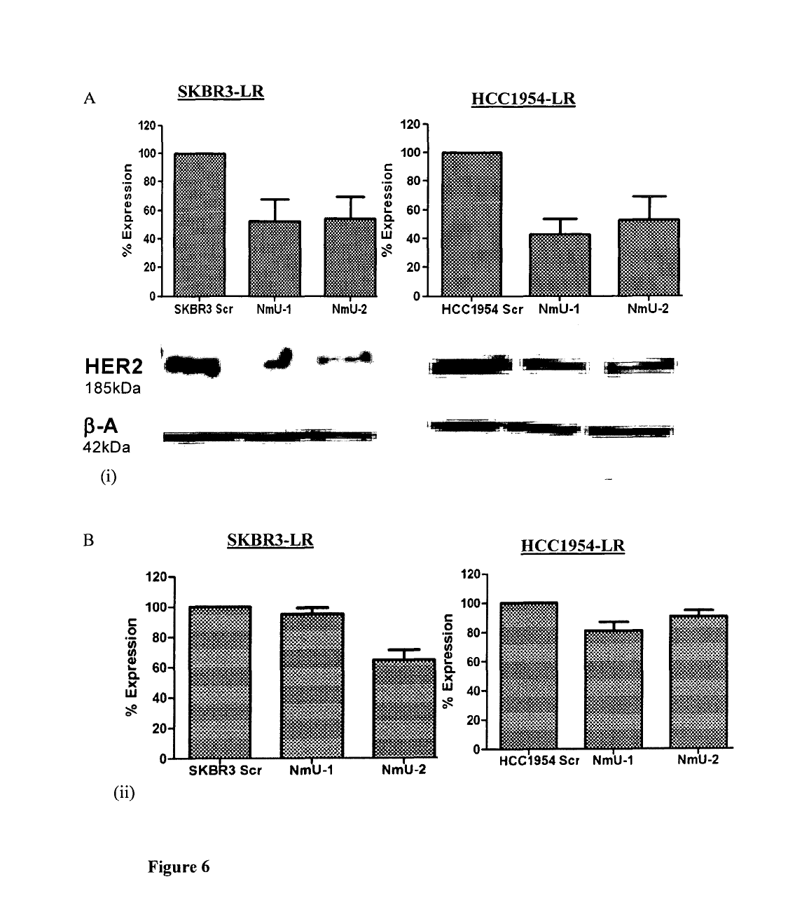

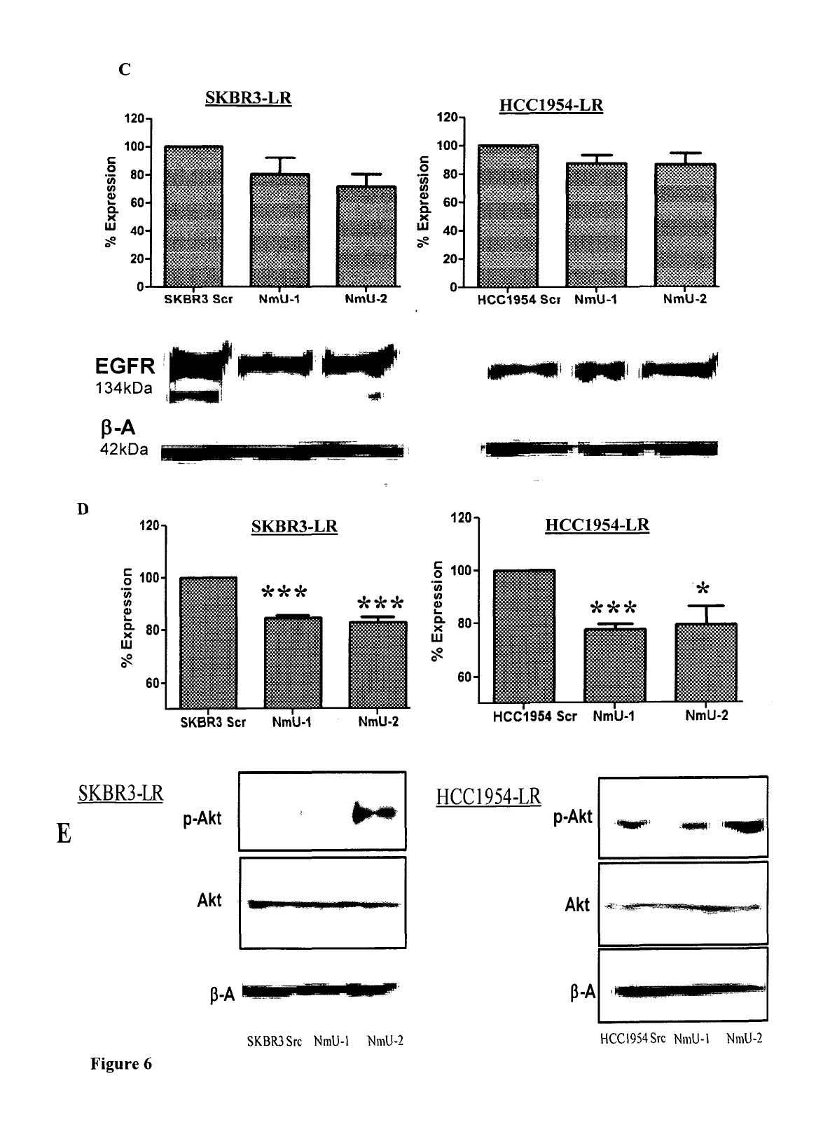

How NmU knock-down may be enhancing the affects of this range of HER-targeted drugs was investigated. As HER2 is a target for all 4 drugs and EGFR is also a target of Lapatinib, Neratinib and Afatinib, the levels of these specific targets—their total amounts and their phosphorylated forms—using ELISAs and immunoblotting were assessed. Of great interest, with both SKBR3-LR and HCC1954-LR cells, NmU knock-down was associated with significantly reduced levels of total HER2 protein (FIG. 6A(i) & (ii)). Knock-down with one siRNA suggested that phosphorylation of the remaining HER2 protein was compromised; however, this observation was not consistent with both siRNA (FIG. 6B) and so not considered as likely to be of relevance. Upon NmU silencing, the total amounts of EGFR present was not significantly affected (FIG. 6C(i) & (ii)), but phosphorylation of the EGFR that was present was significantly reduced (FIG. 6D).

Changes in intracellular signaling through the phosphoinositide-3-kinase(PI3K)/Akt pathway have been associated with resistance to some HER-targeted drugs. For example, PI3K pathway activation has been reported to result in low efficacy of both Lapatinib and Trastuzumab, while transfection of constitutively active Akt into cells has been found to reduce their Lapatinib sensitivity, with kinase-dead Akt increasing sensitivity. Here it was observed that in NmU knock-down cells (which correlated with increased sensitivity to all 4 drugs evaluated), there was significantly increased activation/phosphorylation of the remaining Akt (FIG. 6E).

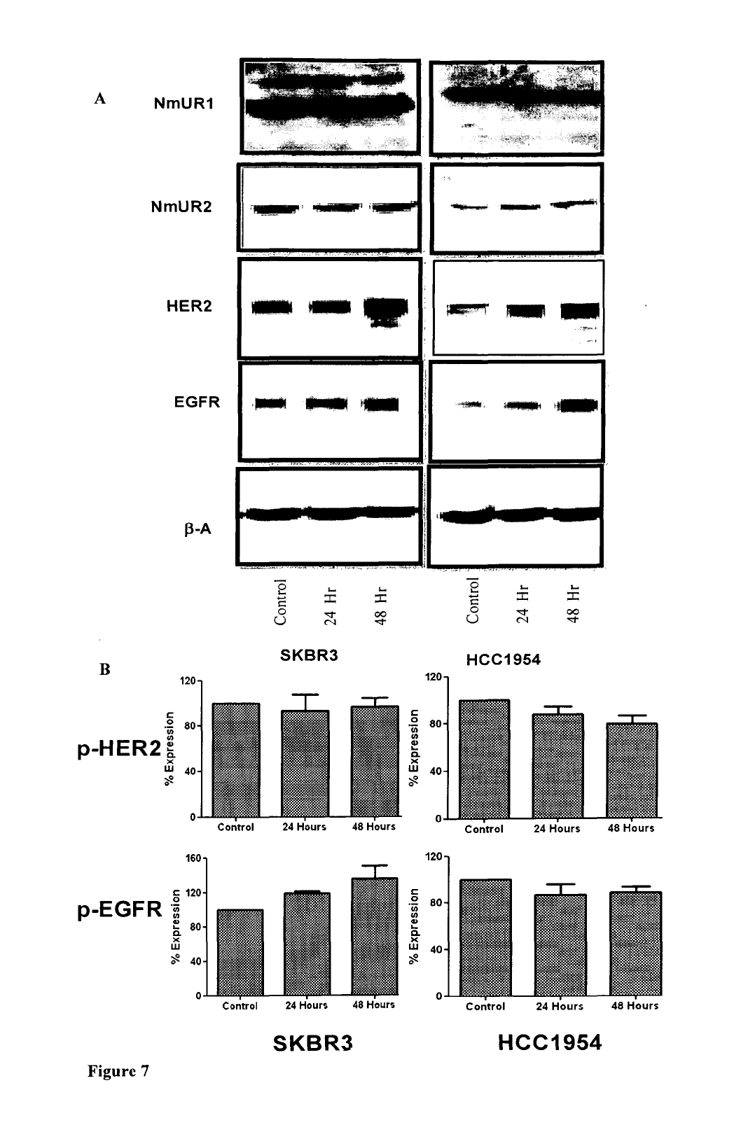

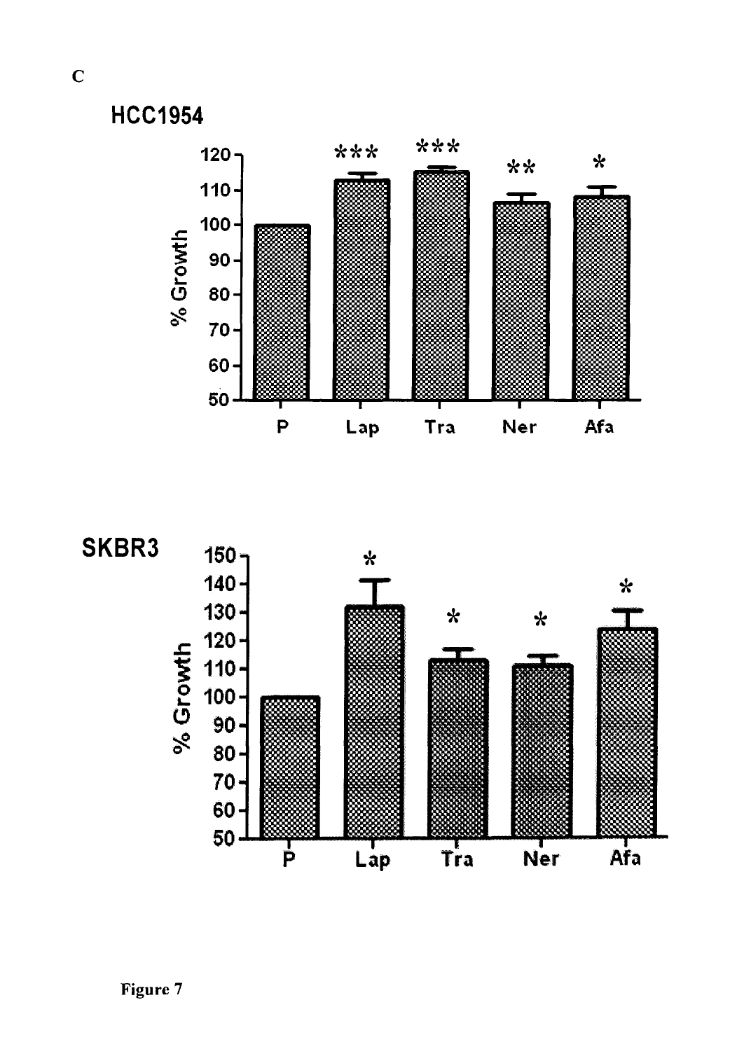

To further explore the functional role of NmU, after establishing that both NmU receptors (NmU-R1, NmU-R2) are expressed by SKBR3 and HCC1954 cells (FIG. 7A), it was observed that treating these cells with exogenous NmU (NmU-25) induced expression of both HER2 and EGFR proteins (and pEGFR in SKBR3 cells; FIG. 7B) suggesting that either or both NmU-R1 and NmU-R2 are functionally active on these cells. Interestingly, this exposure to exogenous NmU also induced a low, but significant, level of resistance to Lapatinib, Trastuzumab, Neratinib and Afatinib in the SKBR3 cells (1.1-1.3 fold) and to a lesser extent in the HCC1954 cells (1.1-fold for all drugs) (FIG. 7C).

NmU Expression is Also Associated with Other Phenotypic Characteristics Including Cell Motility, Invasion and Resistance to Anoikis

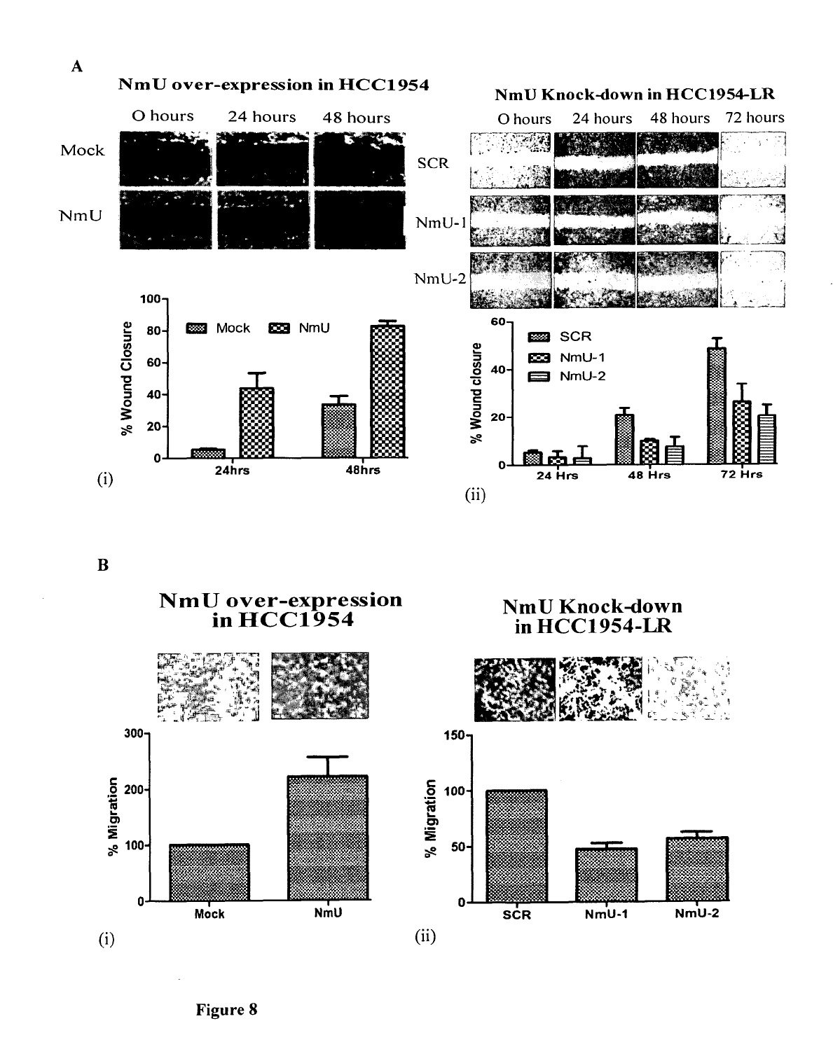

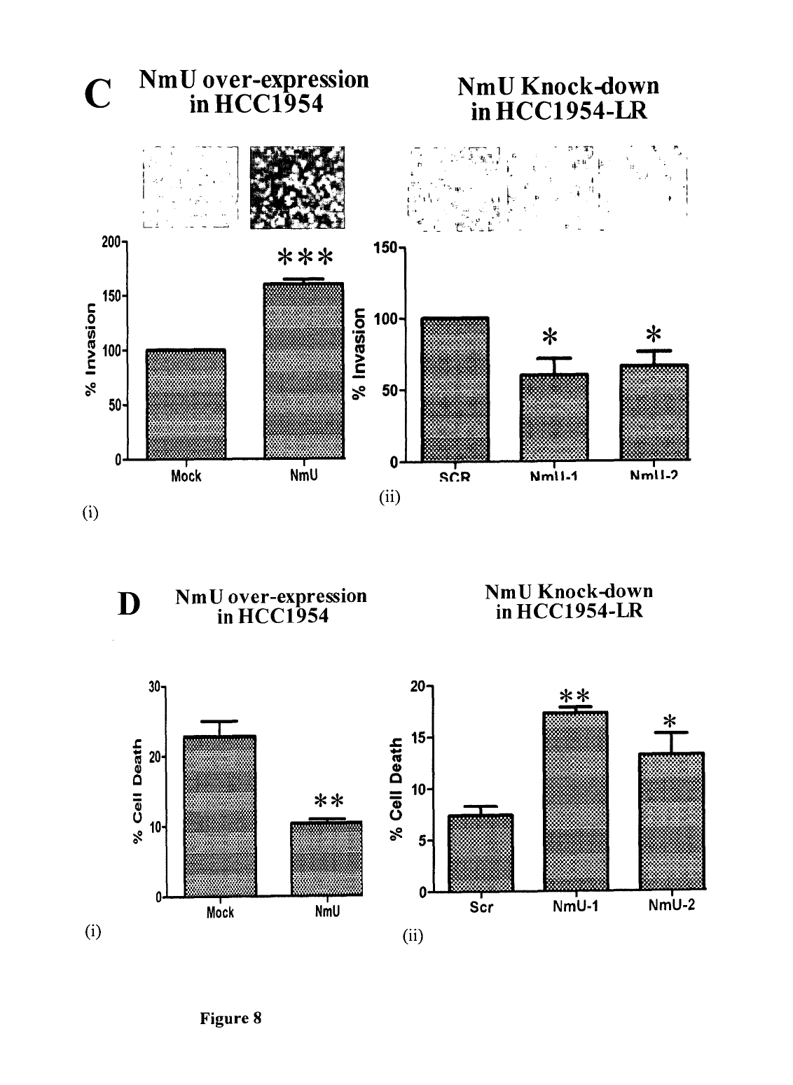

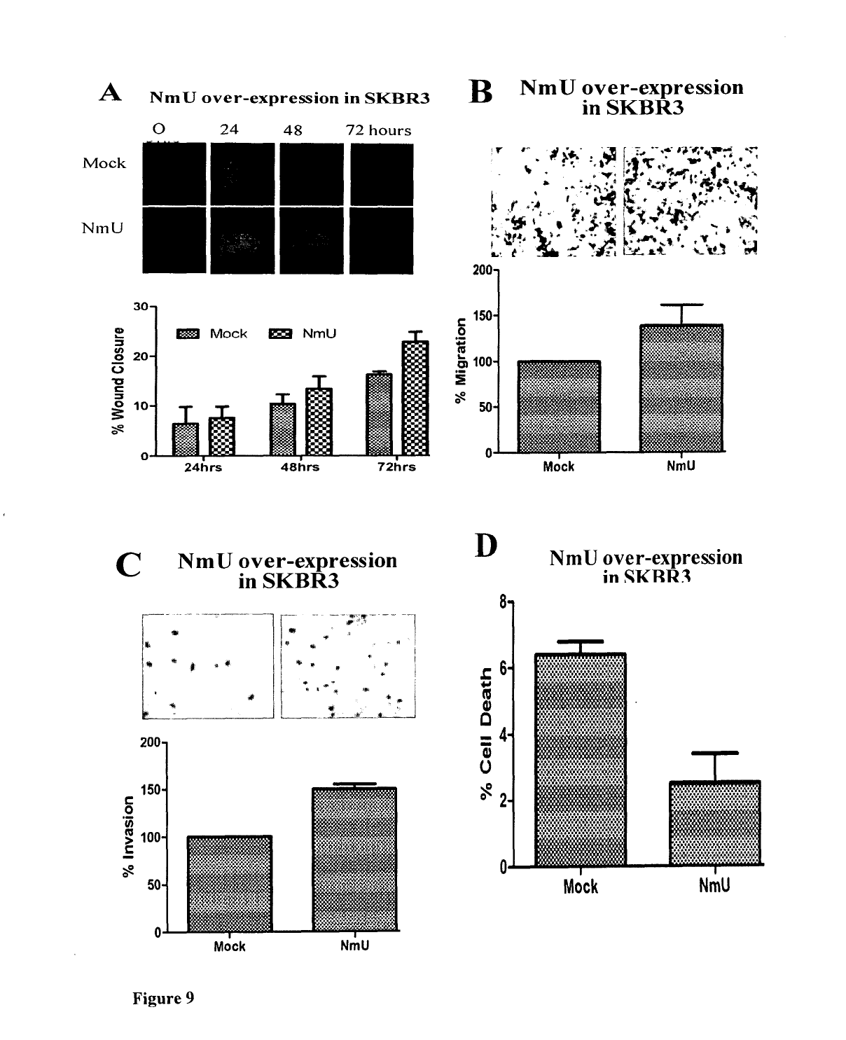

To assess what other functional role(s) NmU may have, NmU-overexpressing and NmU knock-down cells were further evaluated. Events associated with more “aggressive” cancers are the ability of the cells to move, to digest and migrate through extracellular matrix (during intravasation and extravasation) and to survive in suspension (as necessary to survive in the peripheral circulation en route to metastasis). NmU over-expression in HCC1954-LR compared to HCC1954 was associated with increased motility as evaluated via wound-heal (FIG. 8A(i)), increased migration through transwell (FIG. 8B(i)), increased invasion through extracellular matrix-coated transwells (FIG. 8C(i)), and resistance to anoikis (FIG. 8D(i)). Conversely, NmU knock-down was associated with opposite effects i.e. decreased cellular motility (FIG. 8A(ii)), decreased migration (FIG. 8B(ii)), decreased invasion (FIG. 8C(ii)), and increased sensitivity to cell death by anoikis (FIG. 8D(i)). (Representative SKBR3 and SKBR3-LR are results summarised in FIG. 9).

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

### REFERENCES

1. Slamon D J, et al. (1989) Studies of the HER-2/neu proto-oncogene in human breast and ovarian cancer. Science 244(4905):707-712.
2. Cobleigh M A, et al. (1999) Multinational study of the efficacy and safety of humanized anti-HER2 monoclonal antibody in women who have HER2-overexpressing metastatic breast cancer that has progressed after chemotherapy for metastatic disease. J Clin Oncol 17(9):2639-2648.
3. Slamon D, et al. (2011) Adjuvant Trastuzumab in HER2-positive breast cancer. N Engl J Med 365(14):1273-1283.
4. Nahta R & Esteva F J (2007) Trastuzumab: triumphs and tribulations. Oncogene 26(25):3637-3643.
5. Anders C K, et al. (2008) Age-specific differences in oncogenic pathway deregulation seen in human breast tumors. PLoS One 3(1):e1373.
6. Bild A H, et al. (2006) Oncogenic pathway signatures in human cancers as a guide to targeted therapies. Nature, Vol 439, pp 353-357.
7. Calabro A, et al. (2009) Effects of infiltrating lymphocytes and estrogen receptor on gene expression and prognosis in breast cancer. Breast cancer research and treatment 116(1):69-77.
8. Chang H Y, et al. (2005) Robustness, scalability, and integration of a wound-response gene expression signature in predicting breast cancer survival. Proc Natl Acad Sci USA, Vol 102, pp 3738-3743.
9. Chin K, et al. (2006) Genomic and transcriptional aberrations linked to breast cancer pathophysiologies. Cancer cell, Vol 10, pp 529-541.
10. Chanrion M, et al. (2008) A gene expression signature that can predict the recurrence of tamoxifen-treated primary breast cancer. Clinical cancer research: an official journal of the American Association for Cancer Research, Vol 14, pp 1744-1752.
11. Desmedt C, et al. (2007) Strong time dependence of the 76-gene prognostic signature for node-negative breast cancer patients in the TRANSBIG multicenter independent validation series. Clinical cancer research: an official journal of the American Association for Cancer Research, Vol 13, pp 3207-3214.
12. Desmedt C, et al. (2009) The Gene expression Grade Index: a potential predictor of relapse for endocrine-treated breast cancer patients in the BIG 1-98 trial. BMC Med Genomics, Vol 2, p 40.
13. Hu Z, et al. (2006) The molecular portraits of breast tumors are conserved across microarray platforms. BMC Genomics, Vol 7, p 96.
14. Ivshina A V, et al. (2006) Genetic reclassification of histologic grade delineates new clinical subtypes of breast cancer. Cancer Res, Vol 66, pp 10292-10301.
15. Kok M, et al. (2009) Comparison of gene expression profiles predicting progression in breast cancer patients treated with tamoxifen. Breast cancer research and treatment 113(2):275-283.
16. Loi S, et al. (2008) Predicting prognosis using molecular profiling in estrogen receptor-positive breast cancer treated with tamoxifen. BMC Genomics, Vol 9, p 239.
17. Ma X J, et al. (2004) A two-gene expression ratio predicts clinical outcome in breast cancer patients treated with tamoxifen. Cancer cell, Vol 5, pp 607-616.
18. Miller L D, et al. (2005) An expression signature for p53 status in human breast cancer predicts mutation status, transcriptional effects, and patient survival. Proc Natl Acad Sci USA, Vol 102, pp 13550-13555.
19. Pawitan Y, et al. (2005) Gene expression profiling spares early breast cancer patients from adjuvant therapy: derived and validated in two population-based cohorts. Breast Cancer Res, Vol 7, pp R953-964.
20. Sabatier R, et al. (2011) A gene expression signature identifies two prognostic subgroups of basal breast cancer. Breast cancer research and treatment 126(2):407-420.
21. Schmidt M, et al. (2008) The humoral immune system has a key prognostic impact in node-negative breast cancer. Cancer Res, Vol 68, pp 5405-5413.
22. Wang Y, et al. (2005) Gene-expression profiles to predict distant metastasis of lymph-node-negative primary breast cancer. Lancet, Vol 365, pp 671-679.
23. Zhang Y, et al. (2009) The 76-gene signature defines high-risk patients that benefit from adjuvant tamoxifen therapy. Breast cancer research and treatment 116(2):303-309.

1. An assay of identifying the responsiveness of a HER2-positive cancer in a patient to a HER2 targeted drug, the method comprising the step of comparing a level of NeuromedinU in a biological sample obtained from the patient with a reference level of NeuromedinU, wherein a level of NeuromedinU that is increased compared to the reference level of NeuromedinU indicates that the HER2-positive cancer has reduced responsiveness to a HER2 targeted drug.

2. An assay according to claim 1, wherein the HER2 targeted drug is selected from Trastuzumab, Lapatinib, Neratinib, and Afatinib.

3. An assay of identifying an aggressive HER2-positive cancer in a patient or of assessing the metastatic potential of a HER2-positive cancer in a patient or of prediction of poor outcome in a patient having a HER2-positive cancer, the method comprising the step of comparing a level of NeuromedinU in a biological sample obtained from the patient with a reference level of NeuromedinU, wherein detection of a level of NeuromedinU that is increased compared to the reference level of NeuromedinU indicates that the HER2-positive cancer is aggressive or that the HER2-positive cancer has greater potential for metastasis than a HER2-positive cancer that does not have an increased level of NeuromedinU or indicates poor outcome for the patient.

4-5. (canceled)

6. An assay as claimed in claim 1 comprising an additional step of assaying a biological sample from the patient for a level of NeuromedinU.

7. An assay as claimed in claim 1 in which the biological sample is selected from HER2-positive cancer cells, blood, or conditioned medium.

8. An assay as claimed in claim 1 in which the HER2-positive cancer is selected from the list consisting of: breast; NSCLC; pancreas; ovarian; colon; kidney; head and neck; stomach; prostate; gliomas; and biologically aggressive forms of uterine cancer.

9. An assay as claimed in claim 3 in which the cancer is a HER2-positive breast cancer.

10. (canceled)

11. A method for the treatment or prevention of a HER2-positive cancer in a patient comprising a step of administering to the patient a therapeutically effective amount of an inhibitor of NeuromedinU.

12. A method as claimed in claim 11 in which the inhibitor is a nucleic acid capable of inhibiting or reducing the expression of NeuromedinU selected from the group consisting of: siRNA; miRNA; ribozyme, anti-sense oligonucleotide.

13-14. (canceled)

15. A method as claimed in claim 11 in which the inhibitor of NeuromedinU comprises an antibody or antibody fragment having specific binding affinity to NeuromedinU peptide.

16. A method as claimed in claim 11 further comprising a step of administering to the patient a therapeutically effective amount a HER2 targeted drug.

17. (canceled)

18. A method as claimed in claim 11 further comprising a step of administering to the patient a therapeutically effective amount of a HER2 targeted drug selected from the group consisting of: Trastuzumab; Lapatinib; Neratinib; Afatinib; and variants thereof.

19-42. (canceled)

43. An assay as claimed in claim 2 in which the biological sample is selected from HER2-positive cancer cells, blood, or conditioned medium.

44. An assay as claimed in claim 2 in which the HER2-positive cancer is selected from the list consisting of: breast; NSCLC; pancreas; ovarian; colon; kidney; head and neck; stomach; prostate; gliomas; and biologically aggressive forms of uterine cancer.

45. An assay as claimed in claim 3 in which the HER2-positive cancer is selected from the list consisting of: breast; NSCLC; pancreas; ovarian; colon; kidney; head and neck; stomach; prostate; gliomas; and biologically aggressive forms of uterine cancer.

46. An assay as claimed in claim 4 in which the HER2-positive cancer is selected from the list consisting of: breast; NSCLC; pancreas; ovarian; colon; kidney; head and neck; stomach; prostate; gliomas; and biologically aggressive forms of uterine cancer.

47. An assay as claimed in claim 6 in which the HER2-positive cancer is selected from the list consisting of: breast; NSCLC; pancreas; ovarian; colon; kidney; head and neck; stomach; prostate; gliomas; and biologically aggressive forms of uterine cancer.

48. An assay as claimed in claim 7 in which the HER2-positive cancer is selected from the list consisting of: breast; NSCLC; pancreas; ovarian; colon; kidney; head and neck; stomach; prostate; gliomas; and biologically aggressive forms of uterine cancer.

49. A method as claimed in claim 12 in which the inhibitor of NeuromedinU comprises an antibody or antibody fragment having specific binding affinity to NeuromedinU peptide.

50. A method as claimed in claim 12 further comprising a step of administering to the patient a therapeutically effective amount of a HER2 targeted drug selected from the group consisting of: Trastuzumab; Lapatinib; Neratinib; Afatinib; and variants thereof.


SEQUENCE LISTING (5 sequences)

SEQ ID NO: 1 - protein, 25 aa, Homo sapiens
FRVDEEFQSPFASQSRGYFLFRPRN

SEQ ID NO: 2 - DNA, 818 bp, Homo sapiens
agtcctgtgtccgggccccgaggcacagccagggcaccaggtggagcaccagctacgcgtggcgcagcgcagcgtccctagcaccgagcctcccgcagccgccgagatgctgcgaacagagagctgccgccccaggtcgcccgccggacaggtggccgcggcgtccccgctcctgctgctgctgctgctgctcgcctggtgcgcgggcgcctgccgaggtgctccaatattacctcaaggattacagcctgaacaacagctacagttgtggaatgagatagatgatacttgttcgtcttttctgtccattgattctcagcctcaggcatccaacgcactggaggagctttgctttatgattatgggaatgctaccaaagcctcaggaacaagatgaaaaagataatactaaaaggttcttatttcattattcgaagacacagaagttgggcaagtcaaatgttgtgtcgtcagttgtgcatccgttgctgcagctcgttcctcacctgcatgagagaagaatgaagagattcagagtggacgaagaattccaaagtccctttgcaagtcaaagtcgaggatattttttattcaggccacggaatggaagaaggtcagcagggttcatttaaaatggatgccagctaattttccacagagcaatgctatggaatacaaaatgtactgacattttgttttcttctgaaaaaaatccttgctaaatgtactctgttgaaaatccctgtgttgtcaatgttctcagttgtaacaatgttgtaaatgttcaatttgttgaaaattaaaaaatctaaaaataaa

SEQ ID NO: 3 - DNA, 19 bp, Artificial Sequence
  siRNA sequence
aaaggttcttatttcatta

SEQ ID NO: 4 - DNA, 19 bp, Artificial Sequence
  siRNA sequence
agatgatacttgttcgtct

SEQ ID NO: 5 - protein, 174 aa, Homo sapiens
MLRTESCRPRSPAGQVAAASPLLLLLLLLAWCAGACRGAPILPQGLQPEQQLQLWNEIDDTCSSFLSIDSQPQASNALEELCFMIMGMLPKPQEQDEKDNTKRFLFHYSKTQKLGKSNVVSSVVHPLLQLVPHLHERRMKRFRVDEEFQSPFASQSRGYFLFRPRNGRRSAGFI